(19) United States
(12) Patent Application Publication
(10) Pub. No.: US 2003/0104570 A1
(43) Pub. Date: Jun. 5, 2003

(54) Triple fusion proteins comprising ubiquitin fused between thioredoxin and a polypeptide of interest

(76) Inventors: Teresa Elisa Virginia Cabezon Silva, Rixensart (BE); Anne-Marie Eva Fernande Delisse, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/296,770

(22) PCT Filed: Jun. 19, 2001
(86) PCT No.: PCT/EP01/06952

(30) Foreign Application Priority Data
Jun. 26, 2000 (GB) 0015619.0
Oct. 30, 2000 (GB) 0026484.6

(51) Int. Cl. C12P 21/02; C12N 1/21; C07H 21/04; C12N 9/50; C12N 15/74

(52) U.S. Cl. 435/69.1; 435/252.33; 435/320.1; 536/23.2; 435/219

(57) ABSTRACT

The present invention relates to novel expression systems, constructs and vectors for use therein, and to the use of the systems to produce recombinant polypeptides suitable for a range of applications, in particular in medicine. The system is characterized by fusion proteins comprising ubiquitin fused between thioredoxin and a polypeptide of interest.

The present invention relates to novel expression systems, constructs and vectors for use therein, and to the use of the systems to produce recombinant polypeptides suitable for a range of applications, in particular in medicine. In particular the invention relates to systems designed to produce, in a bacterial host, high levels of soluble and conformationally correct recombinant polypeptides. More particularly, the invention relates to the expression of a recombinant polypeptide in the form of a ubiquitin fusion or in the form of a thioredoxin-ubiquitin fusion, and optionally produced with the co-expression of a ubiquitin-specific endoprotease, so as to increase the stability and the degree of the solubility of the desired protein.

Many heterologous proteins are frequently expressed at high levels in bacterial hosts, characterised by fast growth of the host cells to high cell densities in an easily defined medium. However, despite the benefits offered, the overproduction of heterologous proteins, especially eukaryotic proteins, in microbial expression systems such as E. coli and Bacillus subtilis often leads to insoluble cytoplasmic aggregates termed “inclusion bodies”, electron dense particles that primarily consist of the recombinant protein and non-reducible polymers thereof.

The production of inclusion bodies may result from protein aggregation, host-mediated protein aggregation, inaccurate or incomplete processing, incorrect protein modifications and improper protein folding. Eukaryotic proteins also contain cysteine residues that are able to form disulfide bonds in order to stabilise their native structure. Proteins with non-native inter- or intramolecular disulfide bonds as well as reduced cysteine residues are found, when expressed in E. coli, in E. coli cytoplasm as inclusion bodies.

To enable the protein to be utilised requires the denaturation of the agglomerate by strong chaotrophic reagents such as 6 M urea or 8 M guanidinium HCl, followed by renaturation. The yield obtained by such process is low. Moreover, very often the renaturation step does not lead to a correctly folded protein, that is to a protein which retains its native three-dimensional conformation. Additionally, the procedures required to develop an efficient refolding process must be designed on a protein-by-protein basis, and the empirical nature of the design of such a process is not compatible with a large-scale production of proteins of industrial or medical importance.

The mechanism by which proteins become well folded and soluble is not fully understood. While all the information necessary for proper folding of a protein is contained within its primary sequence, experimental evidence support the view that other factors such as protein foldases and isomerases, aid in protein folding and tertiary structure formation.

In E. coli for example, several parameters appear to affect the solubility of eukaryotic proteins, especially when high-expression levels are achieved. For example, the E. coli heat shock chaperone GroESL has been shown to mediate the correct folding of a newly synthetised polypeptide (Weissman, J. S., Kashi, Y., Fenton, W. A. and Horwich, A. L., 1994, Cell 78, 693-702). Additionally, the redox environment between E. coli and eukaryotic cells differs and thus affects the solubility of the protein. Finally, if a protein is itself incorrectly folded, this can affects its solubility, and therefore an incorrectly folded protein often accumulates in the form of insoluble aggregates.

It is therefore often desirable to overcome these fundamental problems of expression and in so doing to maximise the expression of the recombinant protein directly in a soluble form.

European Patent Application No. EP 0 768 382 A2 discloses a method for producing a soluble protein from bacteria, characterised in that the desired protein is co-expressed in trans with thioredoxin (Trx).

Other proteins have been used for fusion protein expression in E. coli, such as Schistosoma japonicum glutathione S-transferase (GST), E. coli maltose-binding protein (MBP) and E. coli DsbA foldase, a periplasmic enzyme involved in disulfide bond formation in proteins (Davis G. D., Elisee C., Newham D. M. and Harrison R. G. 1999, Biotechnology and Bioengineering 65, 382-388).

However, many such fusion proteins, irrespective of the protein origin (prokaryotic or eukaryotic) fail to retain all the biological properties of the target protein. To overcome this, the protein of interest must then be selectively separated from the fusion partner, through an in vitro enzymatic cleavage with a peptidase. This process is inefficient resulting in low yields of the protein of interest and moreover is often not reproducible, providing different cleavage products from run to run. These drawbacks detract from the usefulness of such a system.

Ubiquitin is a highly conserved small (76-amino acid) eukaryotic protein (Butt et al. 1989, Proc. Natl. Acad. Sci. U.S.A. 86, 2540-2544). It is expressed from naturally occurring gene fusions to either itself (i.e. polyubiquitin), or to one or two ribosomal proteins (Baker R. T. 1996, Curr Opin Biotechnol 7, 541-546). While linear fusions with ubiquitin have been reported to have an increased solubility, this is not always the case (R. Baker, S. Smith, R. Marano, J. McKee & P. Board 1994, J. Biol. Chem. 269,25381-25386).

Ubiquitin fusion proteins appear to be rapidly cleavable, precisely after the carboxy-terminal glycine residue of ubiquitin, by members of a large family of ubiquitin-specific proteases (UBP). In Saccharomyces cerevisiae, ubiquitin is generated exclusively by proteolytic processing of precursors in which ubiquitin is either joined to itself, as a linear polyubiquitin protein, or to an unrelated amino acid sequence as a hybrid protein. A family of four ubiquitin-coding loci in the yeast Saccharomyces cerevisiae has been described (E. Ozkaynak, D. Finley, M. Salomon & A. Varshavsky, 1897, The EMBO journal 6, 1429-1439). When ubiquitin fusion proteins are expressed in yeast, endogenous UBPs rapidly and quantitatively remove ubiquitin from the fusion protein (C. Gilchrist, D. Gray & R. Baker 1997, J. Biol. Chem. 272, 32280-32285). Ubiquitin specific proteases cleave G₇₆-X peptide bonds at the carboxyl terminus of the ubiquitin moiety in linear fusions, irrespective of its size or the amino acid residue immediately following ubiquitin, with the single exception of proline (in which case the rate of cleavage is twenty times slower). Members of a large family of UBP enzymes in yeast perform this cleavage. Four UBPs have been cloned and functionally characterized from yeast. Ubiquitin fusion proteins expressed in E. coli remain uncleaved, due to the lack of endogenous UBP activity. The availability of cloned UBP genes has enabled their co-expression in E. coli with ubiquitin fusion proteins. UBP1 as described in WO 91/17245 was shown to have a versatile co-translational cleavage activity, tested in E. coli against fusions of varying size.

The UBP1 proteases remove the ubiquitin moiety from any fusion protein between ubiquitin and a polypeptide, peptide or protein other than ubiquitin. Neither ubiquitin nor ubiquitin-specific proteases are found in E. coli, or for that matter in prokaryotes. Currently, the production of a desired polypeptide from a ubiquitin fusion protein requires the introduction of an in vitro digestion step with a ubiquitin-specific protease. Such a process requires long reaction times and produces variable yields of the desired recombinant product. These disadvantages have impaired the implementation of such a process at industrial scale.

The present invention provides a novel expression system that does not suffer from the drawbacks mentioned above.

In a first aspect, the invention pertains to new fusion proteins between ubiquitin and a target recombinant protein. In another aspect, the invention pertains to fusion proteins wherein the fusion is a “sandwich” ubiquitin fusion protein in which the ubiquitin moiety is positioned between thioredoxin and a polypeptide, peptide or protein of interest. In a further embodiment of the invention is provided a method of producing a protein as described herein.

The protein may be from bacterial, viral, protozoan, fungal and mammalian sources. In particular, the invention relates to fusion proteins between ubiquitin and a tumour-associated antigen, or a differentiation antigen suitable for cancer treatment. Preferred antigens are selected from a group of proteins containing Mage proteins and Mage derivatives thereof, PS108 (WO 98/50567), P501S (WO 98/37418) and derivatives thereof, and prostate cancer-associated protein, prostase and derivatives thereof, disclosed in Ferguson, et al. (Proc. Natl. Acad. Sci. USA 1999, 96, 3114-3119) and in International Patent Applications No. WO 98/12302, WO 98/20117 and WO 00/04149 (P703P). Preferred P501S derivatives include the 55-553 carboxy-terminal end of the protein, preferably the 1-320 amino-terminal and of the protein. Other preferred antigens include Cripto (U.S. Pat. No. 5,256,643), Prame (WO 96/10577), C74_{—}39 (PCT/EP01/01779), C76_{—}1 (GB 0017512.5), viral antigens such as Human Papilloma Virus (HPV) E7, E6 and E2 proteins and derivatives thereof such as ProteinD1/3 E7 (WO 99/10375) and HBV polymerase (Ji Hoon Jeong et al, 1996, BBRC 223, 264271; Lee H. J. et al, Biotechnol. Lett. 15, 821-826).

In another aspect, the invention pertains to fusion proteins wherein the fusion is a “sandwich” ubiquitin fusion protein in which the ubiquitin moiety is positioned between thioredoxin and a polypeptide, peptide or protein of interest. The three terms used hereabove—polypeptide, peptide or protein—can be used interchangeably herein, with peptides usually referring to relatively short polypeptides, on the order of about 50 residues or less. In a specific embodiment, the target recombinant protein within the trifusion is a cancer associated antigen, more preferably the protein is selected from a group of proteins containing Mage proteins and Mage derivatives thereof, PS108 (WO 98/50567) and derivatives, Cripto (U.S. Pat. No. 5,256,643), Prame (WO 96/10577), C74_{—}39 (PCT/EP01/01779), C76_{—}1 (GB 0017512.5), P501S (WO 98/37418) and derivatives thereof, prostase and derivatives thereof (Proc. Natl. Acad. Sci. USA 1999, 96, 3114-3119) and in International Patent Applications No. WO 98/12302, WO 98/20117 and WO 00/04149 (P703P). Preferred P501S derivatives include the 55-553 carboxy-terminal end of the protein, preferably the 1-320 amino-terminal and of the protein. In another specific embodiment the target protein is HPV E2, E7, E6 proteins or fusions therof such as E6E7 fusion, and derivatives thereof such as ProteinD1/3 E7 (WO 99/10375). In a further specific embodiment the target protein is HBV polymerase. The triple fusion combines the ability of the first fusion partner, thioredoxin, in refolding disulfide-containing residues and producing soluble fusion proteins, to the ability of the second fusion partner, ubiquitin, in acting as a chaperone through the stabilisation of the nascent polypeptide chain and in contributing to the recovery of a processed protein through the cleavage site recognised by a specific ubiquitin protease.

Co-translational expression of thioredoxin and ubiquitin increases the solubilisation of the proteins of the invention and has also a significant positive impact on protein purification yield, on purified-protein solubility, stability and quality.

The ubiquitin moiety of the invention is preferably from, but not exclusively limited to, human origin.

A preferred target polypeptide is one from Mage family. Antigens encoded by the family of MAGE genes are predominately expressed on melanoma cells (including malignant melanoma) and some other cancers including NSCLC (non small cell lung cancer), head and neck squamous cell carcinoma, bladder transitional cell carcinoma, oesophagus carcinoma, breast carcinoma and colon carcinoma, but are not detectable on normal tissues except in the testis and the placenta (Gaugler, 1994; Weynants, 1994; Patard, 1995).

Mage family is a family of 12 closely related genes, MAGE 1, MAGE 2, MAGE 3, MAGE 4, MAGE 5, MAGE 6, MAGE 7, MAGE 8, MAGE 9, MAGE 10, MAGE 11, MAGE 12, located on chromosome X and sharing with each other 64 to 85% homology in their coding sequence (De Plaen, 1994). These are sometimes known as MAGE A1, MAGE A2, MAGE A3, MAGE A4, MAGE A5, MAGE A6, MAGE A7, MAGE A8, MAGE A9, MAGE A 10, MAGE A11, MAGE A 12, and currently forming the MAGE A family. Two other groups of proteins are also part of the MAGE family although more distantly related. These are the MAGE B and MAGE C group. The MAGE B family includes MAGE B1 (also known as MAGE Xp1, and DAM 10), MAGE B2 (also known as MAGE Xp2 and DAM 6), MAGE B3 and MAGE B4. The MAGE C protein currently includes MAGE C1 and MAGE C2. In general terms, a MAGE protein can be defined as containing a core sequence signature located towards the C-terminal end of the protein (for example with respect to MAGE A1 a 309 amino acid protein the core signature corresponds to amino acid 195-279). In general a MAGE protein will be approximately 50% identical in a core region with amino acids 195 to 279 of MAGE A1, as described in WO 99/40188. Mage derivatives are also contemplated in the present invention, and are for example described in WO 99/40188.

Another preferred polypeptide is one from a human-papilloma virus that find utility in the treatment or prophylaxis of human papilloma induced tumours. In particular the polypeptide is an early protein, from E1 to E7, most preferably a fusion protein comprising an E2, E6 or E7 protein from HPV strain 16 or 18 linked to protein D from Heamophilius influenza B, as described in WO 99/10375. After integration of viral DNA, the oncogenic process starts, and leads to overexpression of the two early proteins E6 and E7 that will lead to a gradual loss of the normal cellular differentiation and to the development of the carcinoma. E6 and E7 overcome normal cell cycle by inactivating major tumor suppressor proteins, p53 and pRB, the retinoblastoma gene product, respectively.

It is a particularly preferred aspect of the present invention that the vaccines comprise a tumour antigen; such vaccines are surprisingly potent in the therapy of cancer such as prostrate, breast, colorectal, lung, pancreatic, renal, ovarian or melanoma cancers. Accordingly, the formulations may contain tumour-associated antigen, as well as antigens associated with tumour-support mechanisms (e.g. angiogenesis, tumour invasion). Additionally, antigens particularly relevant for vaccines in the therapy of cancer also comprise Prostate-specific membrane antigen (PSMA), Prostate Stem Cell Antigen (PSCA), tyrosinase, survivin, PRAME (WO 96/10577), Cripto (U.S. Pat. No. 5,256,643), NY-ESO1, RAGE, LAGE, HAGE, prostase (P. Nelson, Lu Gan, C. Ferguson, P. Moss, R. Gelinas, L. Hood & K. Wand, Proc. Ntl. Acd. Sci. USA (1999) 96, 3114-3119) (see also WO 98/12302 and corresponding U.S. Pat. No. 5,955,306; WO 98/20117 and corresponding U.S. Pat. No. 5,840,871 and U.S. Pat. No. 5,786,148; and WO 00/04149), PS108 (WO 98/50567), P501S (WO 98/37093 SEQ ID NO: 113) and prostate cancer-associated protein (WO 99/67384 SEQ ID NO: 9). Preferred P501S derivatives include the 55-553 carboxy-terminal end of the protein, preferably the 1-320 amino-terminal and of the protein. These antigens and derivatives thereof are preferred targets polypeptides for expression using the trifusion system.

In a preferred embodiment, the thioredoxin is located upstream of the ubiquitin in the fusion protein. The polypeptide of interest is positioned downstream of the ubiquitin in the fusion protein. In a preferred embodiment, the polypeptide of interest within the triple fusion is selected from the group comprising Mage-3, PS108, P501S and prostate cancer-associated antigen, prostase, protein D E7, and fragments and homologues thereof.

The fusion proteins of the invention may be expressed in unicellular hosts such as prokaryotic and lower eukaryotic organisms, such as yeast and bacteria The fusion proteins of the invention are preferably expressed in E. coli. In a preferred embodiment, the proteins are harbouring an affinity peptide. Preferably the affinity tag comprises a Histidine tail, fused at the carboxy-terminus of the proteins of the invention, preferably comprising between 5 to 8 histidine residues, preferably at least 4 residues, and most preferably 6 histidine residues. Preferably the affinity peptide has adjacent histidine residues, preferably at least two, more preferably at least 4 residues. Most preferably the protein comprises 6 directly neighbouring histidine residues. These histidine tag are designed in aiding the purification of the recombinant protein, particularly by Ni chelate based IMAC chromatography. In another preferred embodiment, the proteins are harbouring a C-LYTA tag at their carboxy-terminus. Preferably the C terminal portion of the molecule is used. Lyta is derived from Streptococcus pneumoniae which synthesize an N-acetyl-L-alanine amidase, amidase LYTA, (coded by the lytA gene {Gene, 43 (1986) page 265-272} an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at its amino terminus has been described {Biotechnology: 10, (1992) page 795-798}. As used herein a preferred embodiment utilises the repeat portion of the Lyta molecule found in the C terminal end starting at residue 178. A particularly preferred form incorporates residues 188-305. These preferential fusions are also new and form one aspect of the invention.

The present invention also provides isolated nucleic acids encoding the fusions of the present invention. More particularly the invention provides for DNA sequences encoding a triple fusion protein comprising ubiquitin fused between thioredoxin and a polypeptide of interest. The invention also provides for DNA sequences encoding a fusion between ubiquitin and at its C-terminus a polypeptide of interest. Codon-optimised genes may be used. Such DNA sequences can be inserted into a suitable expression vector and expressed in an appropriate host cell. In a preferred form of the invention, the expression is carried out in a bacterial strain, most preferably E. coli. The expression vector containing a DNA sequence encoding the fusion according to the invention and the host transformed with said sequence also form part of the invention.

The fusion proteins as described hereabove can be generated using standard DNA synthesis techniques, such as by enzymatic ligation as described by D. M. Roberts et al. in Biochemistry 1985, 24, 5090-5098, by chemical synthesis, by in vitro enzymatic polymerization, or by PCR technology utilising for example a heat stable polymerase, or by a combination of these techniques. Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biemat, J. McMannus, and H. Koester, Nucleic Acids Research, 1984, 12, 4539; and H. W. D. Matthes et al., EMBO Journal, 1984, 3, 801.

The DNA fragments encoding the polypeptide of interest and, in the triple fusion, the thioredoxin partner are ligated to the 3′ end and 5′ end, respectively, of a DNA sequence encoding the ubiquitin. These coding sequences must be joined in frame such that no stop codon is created which would result in the premature termination of the translation of the mRNA encoding the fusion.

In a further embodiment of the invention is provided a method of producing a protein as described herein. In particular, the process of the invention may preferably comprise the steps of:

(a) culturing a host cell transformed with the DNA sequence encoding the fusion protein of the invention, and

(b) recovering the fusion protein.

The final product is preferably recovered in a form that is proteolytically cleavable by a ubiquitin-specific endoprotease. Preferably the ubiquitin-specific endoprotease is the 809-residues protein UBP1, encoded by the UBP1gene, or functional derivatives thereof.

The term ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest. Preferably recombinant antigens of the invention are expressed in unicellular hosts, most preferably in bacterial systems, most preferably in E. coli.

In a preferred aspect of the invention the method is characterised in that the synthetic fusion protein is co-expressed in E. coli with a ubiquitin-specific endoprotease in trans. Co-expression of the ubiquitin-specific endoprotease in trans is preferred to generate a target polypeptide antigen free of thioredoxin and ubiquitin fusion partners, without the need for the addition of a protease in vitro after purification. Accordingly, the methodology of this invention can be used to artificially generate authentic amino-termini in the target polypeptide, or alternatively any amino-terminus of choice. Preferably the ubiquitin-specific endoprotease is UBP1 from Saccharomyces cerevisiae.

In particular, the method of producing a recombinant polypeptide of interest with an authentic amino-terminus may comprise the steps of:

(a) culturing a host under conditions which allow for the co-expression of the fusion of the invention and of the ubiquitin-specific endoprotease and

(b) recovering the recombinant polypeptide of interest, with a defined amino-terminus, after it has been subjected to the action of the ubiquitin-specific protease in vivo.

Preferably the recombinant strategy includes cloning a gene construct encoding a fusion protein, the gene construct comprising from 5′ to 3′ a DNA sequence encoding a thioredoxin joined to a DNA sequence encoding a ubiquitin joined to a DNA sequence encoding a peptide of interest, into an expression vector to form a DNA fragment encoding a thioredoxin-ubiquitin carboxyl-terminal fusion protein. An affinity tag, preferably a polyhistidine tail or a c-LYTA tag, may be engineered at the carboxy-terminus of the fusion protein allowing for simplified purification through affinity chromatography.

The method also includes transforming said expression vector into a suitable bacterial expression strain, preferably bacterial, most preferably E. coli, and allowing the expression of the thioredoxin-ubiquitin carboxyl-terminal fusion polypeptide. The gene construct is preferably under the control of an inducible promoter such as λpL promoter, and the addition of tryptophane to the culture medium allows for the induction of λpL promoter at any temperature. This further improved system may be used to evaluate and monitor at the fermentation level, such as the physiological conditions under which the protein can be better expressed in a essentially more soluble form. The improvement could be observed at the level of acellular extract preparation, where the recombinant protein is predominantly found in the soluble fraction as defined by the standard methods preparation of such extracts followed by standard analysing methods. One such method consists in running a SDS-PAGE with the expressed material (both the pellet and supernatant fractions), followed by Coomassie blue staining, scanning and analysis of the scans by imaging densitometry.

When co-expression of the ubiquitin-specific protease is required, which is a preferred aspect of the invention, the recipient bacterial strain is co-transformed with a compatible so-called processing vector encoding a ubiquitin-specific endoprotease. The term ‘co-transforming’ is used herein to mean the introduction into a suitable host cell of foreign DNA from two compatible plasmids. This can be achieved for example by expressing the protease under the control of a constitutive promoter or an inducible promoter [IPTG]. The ubiquitin protease gene is preferably ubiquitin-protease 1 (UBP1) of Saccharomyces cerevisiae. Alternatively, the ubiquitin protease expression cassette can be placed in the same plasmid than the trimera fusion and could be expressed either under the control of a different promoter, such as a constitutive promoter or under the control of an inductible promoter. The host cell, co-transformed with a DNA sequence encoding a ubiquitin-specific endoprotease also forms part of the invention.

The expression and processing vectors are novel and also form part of the invention. More particularly, the invention includes the recombinant DNA vector containing the Thioredoxin and the Ubiquitin moieties upstream of a polylinker suitable for further cloning of the polypeptide of interest.

The replicable expression vectors may be prepared in accordance with the invention, by cleaving a vector compatible with the host cell to provide a linear DNA segment having an intact replicon, and combining said linear segment with one or more DNA molecules which, together with said linear segment, encode the desired product, such as the hybrid DNA sequence encoding the protein of the invention, or derivative thereof, under ligating conditions.

Thus, the hybrid DNA sequence may be preformed or formed during the construction of the vector, as desired.

The choice of vector will be determined in part by the host cell, which may be prokaryotic or eukaryotic but preferably is E. coli. Suitable vectors include plasmids, bacteriophages, cosmids and recombinant viruses. Expression and cloning vectors preferably contain a selectable marker such that only the host cells expressing the marker will survive under selective conditions. Selection genes include but are not limited to the one encoding protein that confer a resistance to ampicillin, tetracyclin or kanamycin.

The preparation of the replicable expression vector may be carried out conventionally with appropriate enzymes for restriction, polymerisation and ligation of the DNA, by procedures described in, for example, Maniatis et al. cited above.

The recombinant host cell is prepared, in accordance with the invention, by transforming a host cell with a replicable expression vector of the invention or with two compatible vectors of the invention under transforming conditions. Suitable transforming conditions are conventional and are described in, for example, Maniatis et al. cited above, or “DNA Cloning” Vol. II, D. M. Glover ed., IRL Press Ltd, 1985. In particular the invention relates to a bacterium co-transformed with both expression vectors as described herein, the two genes being expressed as two distinct products.

The bacterial strain that is co-transformed with the two compatible vectors of the invention is also part of the present invention.

The choice of transforming conditions is determined by the host cell. Thus, a bacterial host such as E. coli may be treated with a solution of CaCl₂ (Cohen et al., Proc. Nat. Acad. Sci., 1973, 69, 2110) or with a solution comprising a mixture of RbCl, MnCl₂, potassium acetate and glycerol and then with 3-[N-morpholino]-propane-sulphonic acid, RbCl and glycerol. Transformation of lower eukaryotic organisms such as yeast cells in culture by direct uptake may be carried out by using the method of Hinnen et al (J. Adv. Enzyme Reg. 1978, 7, 1929).

Culturing the transformed host cell under conditions permitting the expression of the DNA sequence is carried out conventionally, as described in, for example, Maniatis et al. and “DNA Cloning” cited above. Thus, preferably the cell is supplied with nutrient and cultured at a temperature below 50° C., preferably between 25° C. and 35° C., most preferably at 30° C. The incubation time may vary from a few minutes to a few hours, according to the proportion of the polypeptide in the bacterial cell, as assessed by SDS-PAGE or Western blot.

The recombinant proteins of the inventions are recovered by conventional methods according to the host cell. Thus, where the host cell is bacterial, such as E. coli it may be lysed physically, chemically or enzymatically and the protein product isolated from the resulting lysate. It is then purified using conventional techniques. The specificity of the expression system may be assessed by western blot using an antibody directed against the polypeptide of interest.

Conventional protein isolation techniques include selective precipitation, adsorption chromatography, and affinity chromatography including a monoclonal antibody affinity column.

When the proteins of the present invention are expressed with a histidine tail (His tag), they can easily be purified by affinity chromatography using an ion metal affinity chromatography column (IMAC) column. These fusions that may be processed in vitro by the action of a ubiquitin-specific protease, or in vivo through co-expression of a ubiquitin-specific endoprotease, give rise to a processed polypeptide of interest. The latter, still harboring its histidine tail, can equally be easily purified by affinity chromatography.

These polypeptides can be purified to high levels (greater than 80% preferably greater than 90% pure as visualised by SDS-PAGE) by undergoing further purification steps. An additional purification step is a Q-Sepharose step that may be operated either before or after the IMAC column to yield highly purified protein. They present a major single band when analysed by SDS PAGE under reducing conditions, and western blot analysis show less than 5% host cell protein contamination.

The present invention also provides a method for producing a vaccine containing the processed protein of the invention, comprising admixing the protein with a pharmaceutically acceptable excipient or carrier, or with a suitable adjuvant or immune response enhancer. Additionally, the present invention provides a method for producing a vaccine comprising producing a fusion protein according to the method described above and formulating said protein with a suitable adjuvant, diluent or other pharmaceutically acceptable excipient.

Vaccine preparation is generally described in Vaccine Design—The subunit and adjuvant approach (Ed. Powell and Newman) Pharmaceutical Biotechnology Vol. 6 Plenum Press 1995. Encapsulation within liposomes is described by Fullerton, U.S. Pat. No. 4,235,877.

The proteins of the present invention are preferably adjuvanted. Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes. Other suitable adjuvant systems include, for example, a combination of monophosphoryl lipid A, preferably 3-de-O-acylated monophosphoryl lipid A (3D-MPL) together with an aluminium salt.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Ribi Immunochem Research Inc. (Hamilton, Mont.) (see U.S. Pat. Nos. 4,436,727; 4,877,611; 4,866,034 and 4,912,094). Another preferred adjuvant for use with the proteins of the present invention is a saponin, preferably QS21 (Aquila Biopharmaceuticals Inc., Framingham, Mass.), which may be used alone or in combination with other adjuvants.

For example, an enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO 96/33739.

A particularly potent adjuvant formulation involving QS21, 3D-MPL & tocopherol in an oil in water emulsion is described in WO 95/17210 and is a preferred formulation.

Another suitable adjuvant is CpG. CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. CpG-containing oligonucleotides may also be used alone or in combination with other adjuvants.

For example, an enhanced system involves the combination of a CpG-containing oligonucleotide and a saponin derivative particularly the combination of CpG and QS21 as disclosed in WO 00/09159. Preferably the formulation additionally comprises an oil in water emulsion and tocopherol.

Other preferred adjuvants include Montanide ISA 720 (Seppic, France), SAF (Chiron, California, United States), ISCOMS (CSL), MF-59 (Chiron), the SBAS series of adjuvants (e.g., SBAS-2 or SBAS4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Other preferred adjuvants include adjuvant molecules of the general formula (I):

HO(CH₂CH₂O)ₙ—A—R

Wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (1), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene4-lauryl ether, polyoxyethylene-35-lauryl ether, and polyoxyethylenae-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (1) above may, if desired, be combined with another adjuvant. For example, a preferred adjuvant combination is preferably with CpG as described in the pending UK patent application GB 9820956.2.

### FIGURE LEGENDS

FIG. 1: Design of the thioredoxin-Ubiquitin-Mage3-His fusion

FIG. 2: Sequence of fusion Thioredoxin-Ubiquitin-MAGE 3 HIS. FIG. 2a displays the nucleotide sequence (SEQ ID N^{o}1), and FIG. 2b the amino acid sequence (SEQ ID N^{o}2).

FIG. 3: Cloning strategy for the construction of pRIT 15021. FIG. 3a shows the cloning strategy, and FIG. 3b the resulting expression plasmid.

FIG. 4: Cloning strategy for the construction of pRIT 15069

FIG. 5: Construction of the plasmid encoding yeast UBP1

FIG. 6: Characterisation of the fusion protein Thioredoxin-Ubiquitin-Mage3 His, of the fusion protein Ubiquitin-Mage3 His and of the in-vivo processing of the recombinantly expressed fusion Thioredoxin-Ubiquitin-Mage3 His.

FIG. 7: Construction of the vector pRIT15088 designed to express recombinant proteins fused to Thioredoxin and Ubiquitin.

FIG. 8: Construction of pRIT15090 a plasmid expressing fusion thioredoxin-ubiquitin-P501 (aa 55→553). FIG. 8a shows the restriction map of pRIT 15063 expressing P501S (55→553), and FIG. 8b the cloning strategy of pRrr 15090.

FIG. 9: Sequence of fusion Thioredoxin-Ubiquitin-P501S (aa 55→553) His. FIG. 9a displays the nucleotide sequence (SEQ ID N^{o}3), and FIG. 9b the amino acid sequence (SEQ ID N^{o}4).

FIG. 10: Characterisation of the fusion protein Thioredoxin-Ubiquitin-P501S (55→553) His and of the in-vivo processing of the recombinantly expressed fusion Thioredoxin-Ubiquitin-P501S (55→553) His. The fusion was expressed by GI724 (pRIT15090) and GI724 (pRIT 15022, pRIT15090) at 30° C.

FIG. 11: Design of the thioredoxin-Ubiquitin-P501S (1-320)-His fusion.

FIG. 12: Sequence of fusion Thioredoxin-Ubiquitin-P501S (1-320) HIS. FIG. 12a displays the amino acid sequence (SEQ ID N^{o}5), and FIG. 12b the mucleotide sequence (SEQ ID N^{o}6). The Thioredoxin and the linker sequences appear in normal font, the ubiquitin sequence appears in italic, and P501S amino acid sequence appears in bold.

FIG. 13: construction of the expression vector pRIT15088 (TCAJ14)

FIG. 14: Construction of pRIT15115 (TCAJ21), a plasmid expressing the fuion Thioredoxin-Ubiquitin-P501S (1-320) His.

FIG. 15: Construction of plasmid pRIT15139 (TCAJ23), a control counterpart expressing P501S(1-320) without fusions partners thioredoxin and ubiquitin.

FIG. 16: Expression of fusion protein Thioredoxin-Ubiquitin-P501S (1-320) by GI724 (pRIT15115) and processed P501S (1-320) by GI724 (pRIT15115, pRIT15022) at 30° C.

FIG. 17: Expression of control counterpart GI724 (pRIT15139) without fusion partners in comparison to fusion protein Thioredoxin-Ubiquitin-P501S (1->320).

FIG. 18: Design of the Thioredoxin-Ubiquitin-ProtD1/3 E7-His fusion.

FIG. 19: Sequence of Thioredoxin-Ubiquitin-ProtD1/3 E7-His fusion. FIG. 19a displays the amino acid sequence (SEQ ID N^{o}7), and FIG. 19b the mucleotide sequence (SEQ ID N^{o}8). The Thioredoxin and the linker sequences appear in normal font, the ubiquitin sequence appears in italic, and ProteinD1/3-E7 amino acid sequence appears in bold.

FIG. 20: Construction of the expression vector pRIT15089 (TCAJ15).

FIG. 21: Construction of pRIT15106 (TCAJ17), a plasmid expressing fusion Thioredoxin-Ubiquitin-ProtD1/3-E7.

FIG. 22: Construction of plasmid pRIT15097 (TCAJ19) a control counterpart expressing ProteinD1/3-E7-His without fusions partners.

FIG. 23: Characterisation of the fusion protein Thioredoxin-Ubiquitin-ProteinD1/3-E7 His and of the in-vivo processing of the recombinantly expressed fusion Thioredoxin-Ubiquitin-ProteinD1/3-E7 His.

FIG. 24: Construction of compatible plasmid expressing ubiquitinase (UBP1) under the controle of TRC promoter. FIG. 24a shows the introduction of ubiquitinase coding sequence under the controle of TRC promoter in vector TRC99A. FIG. 24b illustrates the transfer of SpH I-Sal I fragment from TCAJ18 (pRIT15116) into pACY184 compatible vector.

The invention will be further described by reference to the following examples.

### EXAMPLE I

### Preparation of the E. coli Strain Expressing the Fusion Protein Thioredoxin-Ubiquitin-Mage3 His

1.—Protein Design

This construction is based on the design of a triple fusion protein, recognized by a ubiquitin protease (UBP1) that liberates the protein of interest in the cytoplasm of E. coli. The fusion protein contains thioredoxin and ubiquitin as fusion partners and Mage 3 as the heterologous protein to express. The thioredoxin gene (trxA) from E. coli comes from a commercial vector from Invitrogen that allows cloning downstream from the trxA gene. The ubiquitin ORF comes from the huan ubiquitin gene. Ubiquitin is a highly conserved protein and therefore human ubiquitin is recognized by UBP1 of Saccharomyces cerevisiae whose cleavage site follows the C terminal glycine76 of Ubiquitin.

Ubiquitin and Mage 3 were cloned, in order, downstream of thioredoxin. Thioredoxin is connected to ubiquitin via a S-G-G-G linker. This linker is added to limit steric interactions between the fusion partners that may hinder their individual effects. The junctional residue between ubiquitin and Mage 3 is a methionine. Modifications were brought to the N-terminus of the natural Mage 3 sequence: the second and third acids P and L, destabilising residues according to the N-end rule, were removed from natural Mage 3 sequence. A Histidine tail was added to Mage 3 to enable versatile purification of the fusion and processed protein.

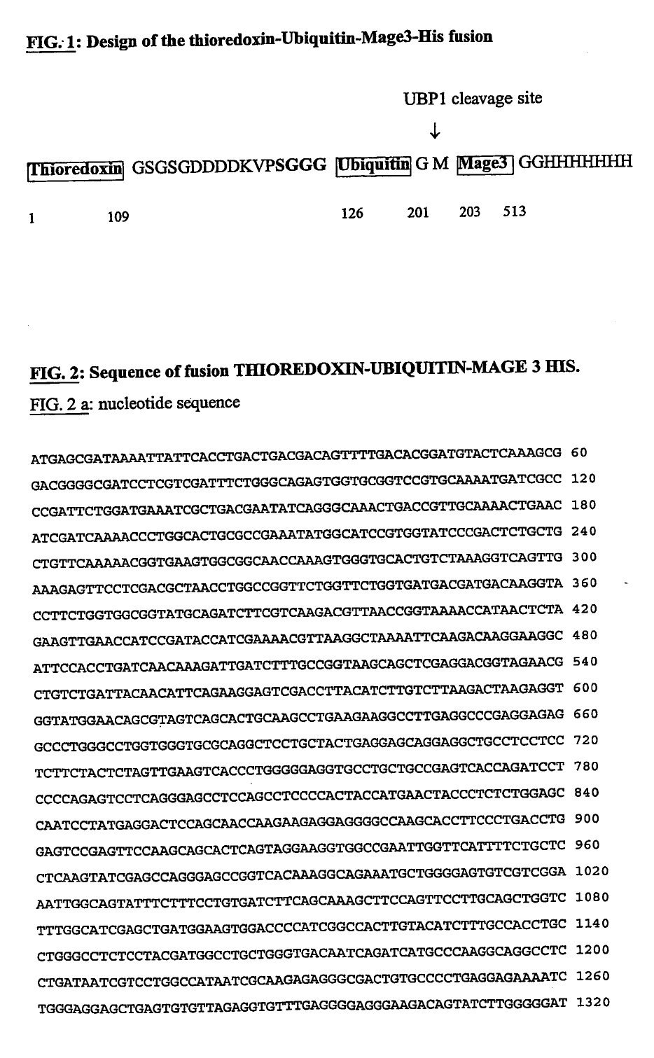

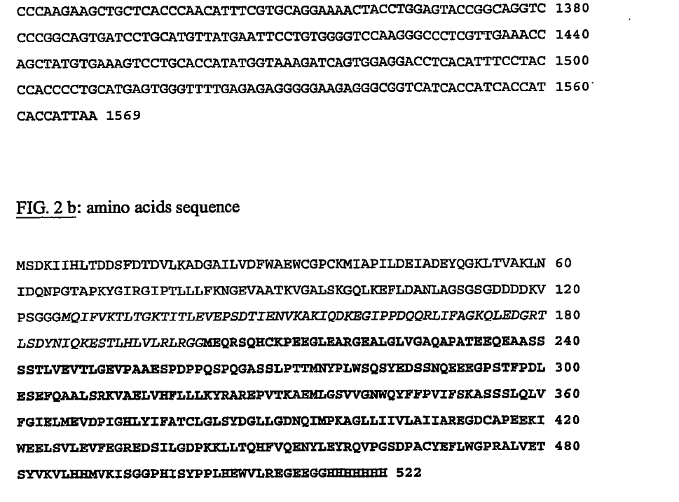

The design of the fusion Thioredoxin-Ubiquitin-Mage3 to be expressed in E. coli is described in FIG. 1. The nucleotide coding sequence corrsponding to the above protein design is depicted in FIG. 2a (SEQ ID N^{o}1), and was placed under the control of λpL promoter in a E. coli expression plasmid. The length of the triple fission is 522 aminoacids and Mage 3 after cleavage gives a protein of 321 aminoacids (in bold in the sequence of FIG. 2b which depicts SEQ ID N^{o}2).

2.—Cloning Strategy for the Generation of the Plasmid pTUbiM3 (=pRIT 15021), a Plasmid Expressing the Fusion Thioredoxin-Ubiquitin-Mage3 His

Several plasmids were used as starting material for the generation of vector pRIT15021 expressing Thioredoxin-Ubiquitin-Mage3 His:

a plasmid pNMHubPoly (5253 bp) received from Dr. Shatzman (SmithKline Philadelphia) containing human ubiquitin coding sequence

a plasmid pRIT14477 (6622 bp) bearing Mage3 coding sequence (described in WO 99/40188);

pTRXfus, an Invitrogen's expression vector used to fuse heterologous proteins with thioredoxin, downstream the λpL promoter.

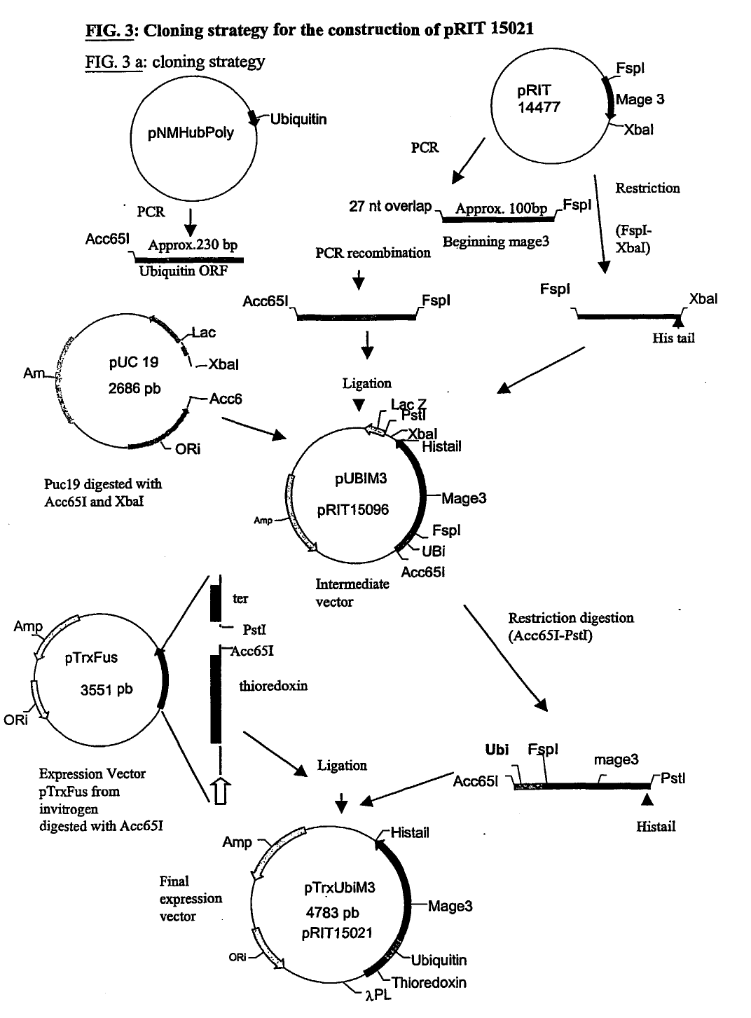

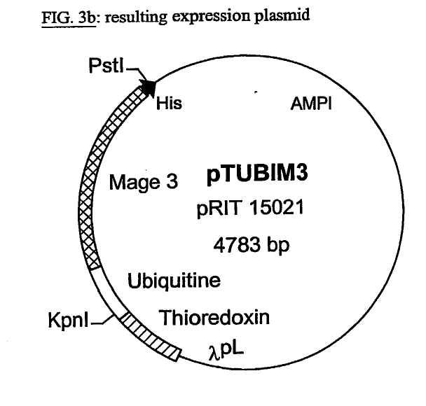

The strategy used to generate the plasmid expressing the triple fusion thioredoxin, ubiquitin and Mage3 in frame with an histidine tail is represented in FIG. 3. It included the following steps.

a)—PCR amplification of ubiquitin sequence with an extension containing a Acc65I restriction site (in italic), a SGGG linker coding sequence (in bold). The template for the PCR reaction was plasmid pNMHubPoly, the forward primer was the 49-mer oligonucleotide [5′-CGG-GGT-ACC-TTC-TGG-TGG-CGG-TAT-GCA-GAT-CTT-CGT-CAA-GAC-GTT-AAC-C-3′] and the reverse primer was the 27-mer oligonucleotide [5′-ACC-ACC-TCT-TAG-TCT-TAA-GAC-AAG-ATG-3′].

b)—PCR amplification of the first 100 bp of Mage 3 sequence containing at the 5′ extremity a 27 nucleotides sequence complementary to the 3′ extremity of the ubiquitin sequence (in bold). The template for the PCR reaction was plasmid pRIT14477 (described in WO 99/40188), the coding primer was the 57-mer oligonucleotide [5′-CAT-CTT-GTC-TTA-AGA-CTA-AGA-GGT-GGT-ATG-GAA-CAG-CGT-AGC-CAG-CAC-TGC-AAG-CCT-3′] and the reverse primer was the 24-mer oligonucleotide containing the FspI restriction site (in italic) present in the Mage 3 sequence [5′-CCT-CAG-TAG-CAG-GAG-CCT-GCG-CAC-3′].

c)—Both PCR fragments were joined by PCR using the 49 mer oligonucleotide forward primer and the 24-mer oligonucleotide reverse primer generating a 330 bp fragment with an ACC65I restriction site at its 5′ extremity and a FspI restriction site at its 3′ extremity. The rest of the Mage 3 ORF was obtained by restriction digestion of TCM222 with FspI and XbaI restriction enzymes.

d)—The intermediate construction was obtained by a three-fragment ligation of the PCR fragment (Acc65I-FspI), the restriction fragment (FspI-XbaI) and pUC19 (Acc65I-Xbal) to give pRIT 15096 (pUBIM3, 3896 bp). The restriction fragment FspI-XbaI was verified by sequencing.

e)—Plasmid pRIT15096 containing the double fusion gene with the correct sequence were digested with Acc65I and PstI restriction enzymes (the ubiquitin ORF contains a XbaI restriction site so XbaI could not be used to extract the fragment from pUC19). The 1205 bp fragment was cloned into pTrxFus digested with Acc65I and PstI to give the final expression plasmid pRIT15021 (pTRXUbiM3)

The resulting plasmid pRIT15021 is used to express the triple fusion between thioredoxin, ubiquitin and Mage3 in frame with a Histidine tail under the control of the inducible λpL promoter (Ampicillin selection).

3.—Transformation of E. coil GI724 strain by pRIT 15021 Leading to E. coli B1285 Strain

Plasmid pRIT 15021 (ampicillin resistant) was introduced by selection for transformants resistant to ampicillin (100 μgr/ml) into E. coli GI724 (F-, λ-, lacIq, lacPL8,ampC::Ptrp,, cI) from Invitrogen.

The E. coli recipient strain GI724 contains an engineered cI repressor gene into the bacterial chromosome under control of the tightly regulated tryptophan promoter, allowing expression of the gene of interest by addition of tryptophan (Mieschendahl et al, (1986) Bio/Technology, 4: 802-808). This can be done at any temperature.

4.—Growth and Induction of the Bacterial Strain B1285

E. coli B1285 strain was grown at 30° C. in induction medium (from Invitrogen) at 30° C. supplemented with convenient antibiotics.

During the logarithmic phase of growth of bacteria, induction at 30° C. was realized by addition of tryptophan (100 μgr/ml final) to repress the production of λ repressor and turn on the synthesis of triple fusion. The incubation at 30° C. was continued for 4 hours. Bacteria were harvested at T=0, T=2 hours and T=4 hours of induction. Bacteria were pelleted and stored at −20° C.

### EXAMPLE II

### Preparation of the E. coli Strain Expressing the Control Counterpart Mage3 His Without Fusion Partner

1.—Cloning Strategy for the Generation of the Plasmid pRIT 15069 (TCAJ12), a Plasmid Expressing the Mage 3-His Without Fusion Partner

Several plasmids were used as starting material for the generation of vector pRIT15069 expressing Mage 3 His:

a plasmid vector pAL781 (Invitrogen) is used as vector control for expression of protein downstream λpL promoter. It does not contain thioredoxin.

a plasmid pRIT 15096, a pUC19-derived plasmid bearing Ubiquitin-Mage 3 coding sequence.

a plasmid pRIT 14477 (TCM222) bearing Mage 3 coding sequence (described in WO 99/40188).

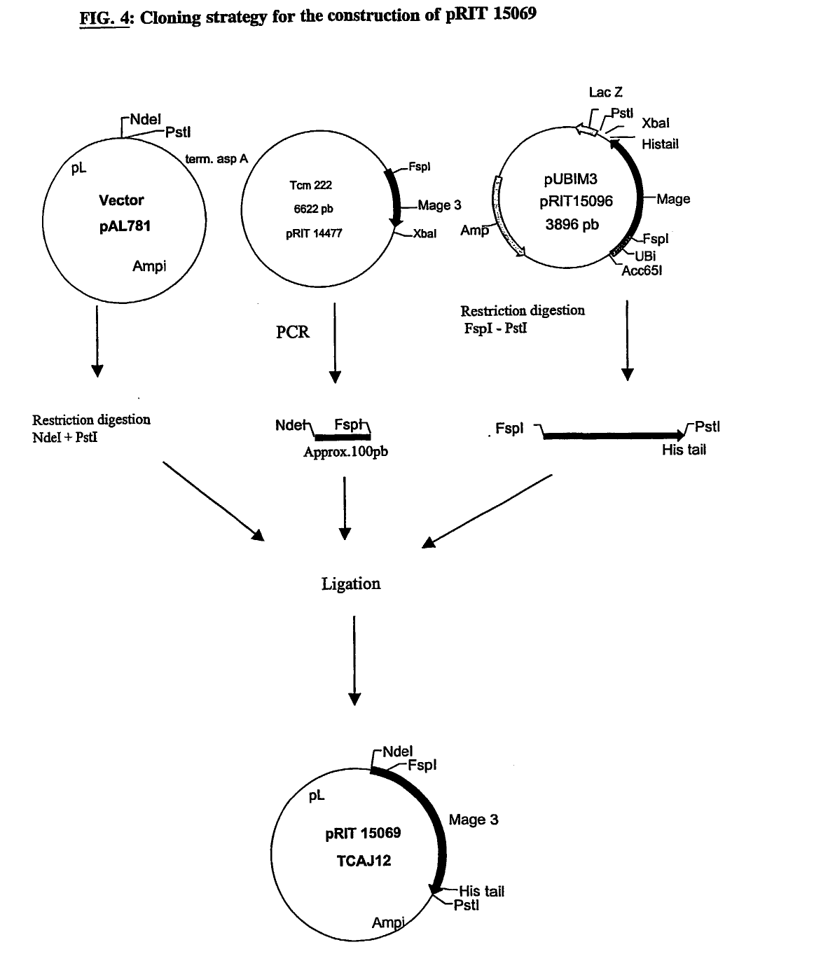

The cloning strategy included the different steps (FIG. 4).

a)—Plasmid vector pAL781 was digested with restriction enzymes NdeI and PstI.

b)—PCR amplification of the first ˜100 bp of Mage 3 sequence with a NdeI restriction site at the 5′extremity and a FspI restriction site at the 3′extremity. The template for the PCR reaction was plasmid pRIT14477, the coding primer was the 34-mer oligonucleotide containing the Nde I restriction site (in italic) [5′-CCA GCA TAT GGA ACA GCG TAG TCA GCA CTG CAA G-3′] and the reverse primer was the 24-mer oligonucleotide containing the FspI restriction site (in italic) [5′-CCT CAG TAG CAG GAG CCT GCG CA C-3′].

c)—The rest of the Mage 3 ORF was obtained by digestion of pRIT15096 with FspI and PstI restriction sites.

d)—Ligation of vector pAL781 digested with NdeI and FspI, with PCR fragment (NdeI-FspI) and restriction fragment (FspI-PstI) was realised to give the expression plasmid pRIT15069 (TCAJ12).

2.—Transformation of E. coli GI724 Strain by pRIT 15069 Leading to E. coli B1326 Strain

Plasmid pRIT 15069 (ampicillin resistant) was introduced by selection for transformants resistant to ampicillin (100 μgr/ml) into E. coli GI724 (F-, λ-, lacIq, lacPL8,ampC::Ptrp,, cI) from Invitrogen. The growth and induction of the bacterial strain B1326 was performed as described in Example I, paragraph 4.

### EXAMPLE III

### Construction of an E. coli Strain Expressing the Fusion Protein Thioredoxin-Ubiquitin-Mage3 His and Co-expressing the Ubiquitin-specific Endoprotease UBP1

1.—Construction of the Plasmid pRIT 15022, a Plasmid Expressing the Ubiquitin-specific Endoprotease UBP1 from Saccharomyces cerevisiae

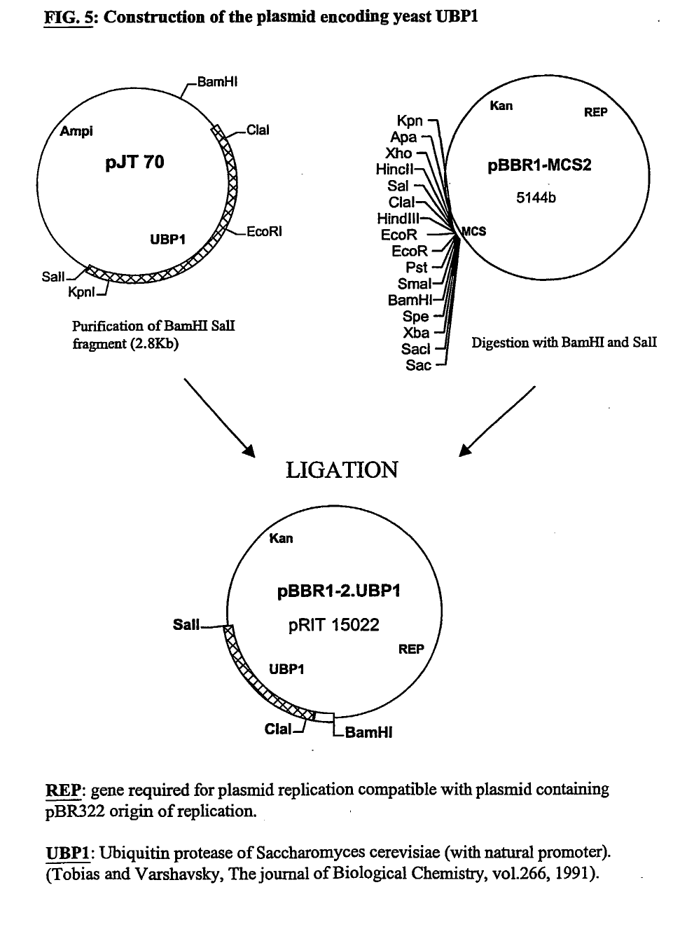

A plasmid (pRIT15022) derived from pBBR1-2, compatible with pRIT15021 and expressing constitutively the Saccharomyces cerevisiae ubiquitin protease (UBP1) (Kanamycin selection) has been engineered (see FIG. 5). The cleavage, in vivo, of the protein of interest from the triple fusion is made possible through co-expression of both plasmid in the recipient E. coli strain.

The starting materials were:

a plasmid pJT70 (Tobias and Varshavsky (1991) J. Biol. Chem, 266,12021-12028) received from Dr Bollen (Université Libre de Bruxelles), containing UBP1gene from Saccharomyces cerevisiae under the control of its own promoter (functioning in E. coli)

a plasmid pBBR1 MCS2, a kanamycin resistant plasmid (M. E. Kovach, R. W. Phillips, P. H. Elzer, R. M. Roop II and K. M. Peterson, 1994, Biotechliques, 16 (5), 800-802), bears an origin of replication of Bordetella bronchiseptica which makes it compatible with ColE1-based vectors derived from pBR322.

The UBP1 complete gene (with its natural promoter, functioning in E. coli) was taken from plasmid pJT70 by restriction digestion with BamHI and SalI. The 2.8 kb fragment was cloned into pBBR1MCS2, to give pRIT15022 (kanamycin resistant).

2.—Transformation of E. coli GI724 Strain by pRIT15021 and pRIT 15022 Leading to E. coli B1286 Strain

The initial E. coli recipient strain GI724 (allowing induction of λpL promoter by tryptophane at any temperature) (F-, λ-, lacIq, lacPL8,ampC::Ptrp,, cI) was co-transformed by plasmid pRIT15021 (ampicillin resistant) and pRIT15022 (kanamycin resistant). The resulting strain harboring both plasmids, was named B1286 and allows the selection through the resistance to the ampicillin (100 μgr/ml) and kanamycin (50 μgr/ml). The growth and induction of B1286 bacterial strain was done according to the conditions described in Example I, paragraph 4.

### EXAMPLE IV

### Characterisation of the Fusion Protein Ubiquitin-Mage 3-His, of the Fusion Thioredoxin-Ubiquitin-Mage 3-His and of the in-vivo Processing of the Recombinantly Expressed Fusion Thioredoxin-Ubiquitin-Mage 3 His

1.—Preparation of Extracts/Analysis on Coomassie Stained SDS-polyacrylamide 12,5% Gels and Western Blots

Frozen cells were thawed and resuspended in PBS buffer. Cells were broken in a cell disrupter One Shot. After centrifugation (20 minutes 16000 g at 4° C.) pellet, supernatant and total extract were analysed by SDS-PAGE. Proteins were visualized on Coomassie blue stained gels and identified by western blot using anti-Mage 3 monoclonal antibodies (clone MG32, SB Biologicals) and anti-His tail monoclonal antibodies (Pentahis of Qiagen).

Determination of percentage and quantity of expressed protein in both supernatants and pellets was carried out on stained gels. Gels were scanned with a Bio-rad GS-700 Imaging Densitometer and data was analyzed by Bio-rad Multi-Analyst™/PC Version 1.1. Specific yield data were generated by taking, as a percent, the ratio of the area under the protein of interest peak over the sum of the areas under all peaks in the same lane.

2.—Results and Conclusions

The experimental approach described above has allowed a comparison of the expression pattern of Mage3 alone, Mage3 tri-fusion form and Mage 3 processed in vivo using the same genetic system (pL promoter, tryptophan induction, 30°).

For the strain control counterpart B1326=GI724 (pRIT 15069) expressing the Mage 3-His without fusion partner, a major band of about 45 KD was detected, slightly visible in Coomassie gels (<1% of total protein) and distributed half in the supernatant and half in the pellet.

For the strain B1285=GI724 (pRIT 15021), a band of 65 KD, corresponding to the triple fusion protein Thioredoxin-Ubiquitin-Mage 3-His was visible in Coomassie gels and mainly present in supernatant.

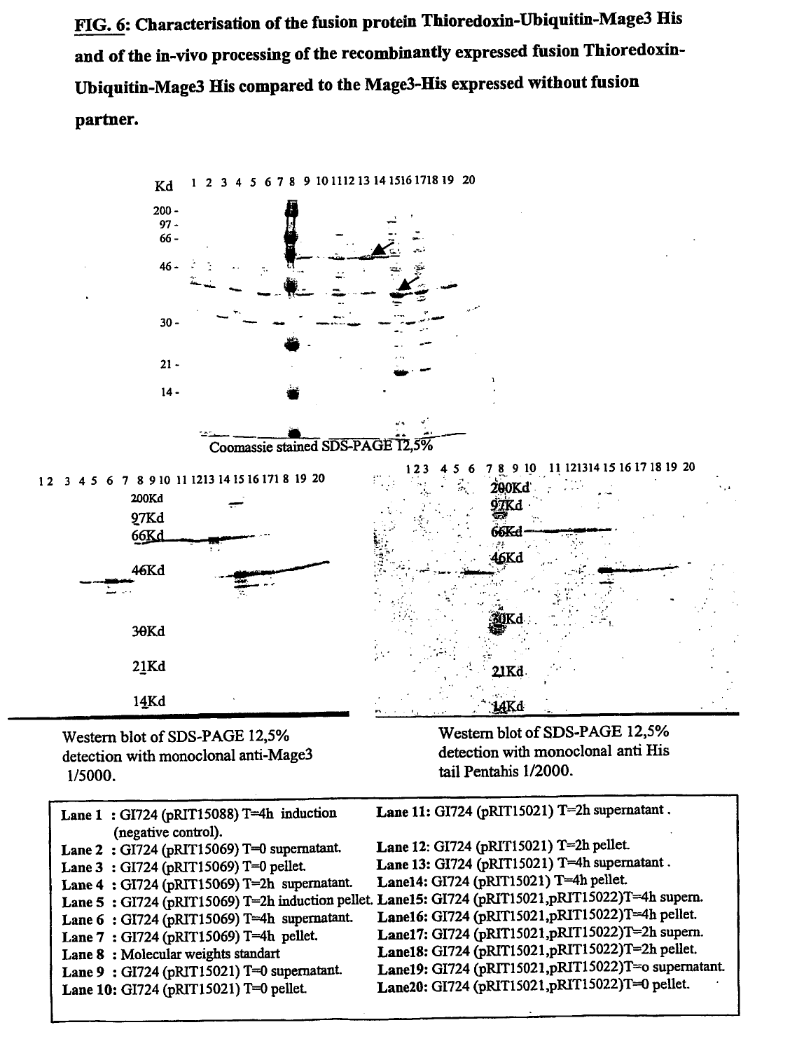

To test for in vivo cleavage of the triple fusion protein, strain B1286=GI724 (pRIT 15021, pRIT 15022) expressing simultaneously the triple fusion and ubiquitinase UBP1 was expressed. Two resulting fragments were obtained, highly visible in Coomassie gels (FIG. 6), found at approximately 45 KD and 25 KD. The 45 KD band, mainly present in supernatant, represents the processed Mage 3 protein as demonstrated by western blot (FIG. 6) and the band of 25 KD corresponds with the cleaved fusion partners, as both moieties are recognized by specific antibodies anti-thioredoxin and anti-ubiquitin.

In previous experiments using heat induced pL promoter for expression of Mage 3 alone and tryptophan induced pL promoter for the triple fusion (the quantity of soluble processed Mage 3 was estimated between 10-20 mg/liter (4% of total proteins), the solubility of the processed Mage 3, ranged from 50% to approximately 80% as assessed by SDS-Page followed by Coomassie blue staining, scanning and analysis of the scans by imaging densitometry. Another experiment has similarly shown that solubility of Mage 3 is significantly increased from a 30-70 supernatant/pellet distribution in the classical heat-induced system to 95-5 distribution in the 30° C. triple fusion system.

In this case, the efficient in vivo processing of the triple fusion protein went along with an increase in yield of more homogeneous, less oxydised and less degraded soluble product of approximately 4-fold in comparison to previous expression results for Mage 3. The recombinant protein liberated in vivo appears more homogenous than the counterpart expressed as a single protein.

### EXAMPLE V

### Construction of an E. coli Strain Expressing the Fusion Thioredoxin-Ubiquitin-P501S (55→553) His

1.—Construction of the Expression Plasmid Expressing the Triple Fusion Thioredoxin-Ubiquitin-P501S (55-553) His

The strategy involves two plasmids. The first plasmid is pRIT15021 (pTUbiM3) coding for the triple fusion thioredoxin-ubiquitin-Mage 3. The second plasmid is pRIT 15063 (ma321) bearing the coding sequence for amino-acids 55 to 553 of prostate antigen P501S [WO 98/37093], elongated with a polyhistidine tail at the carboxy-terminus.

The cloning strategy involved three steps:

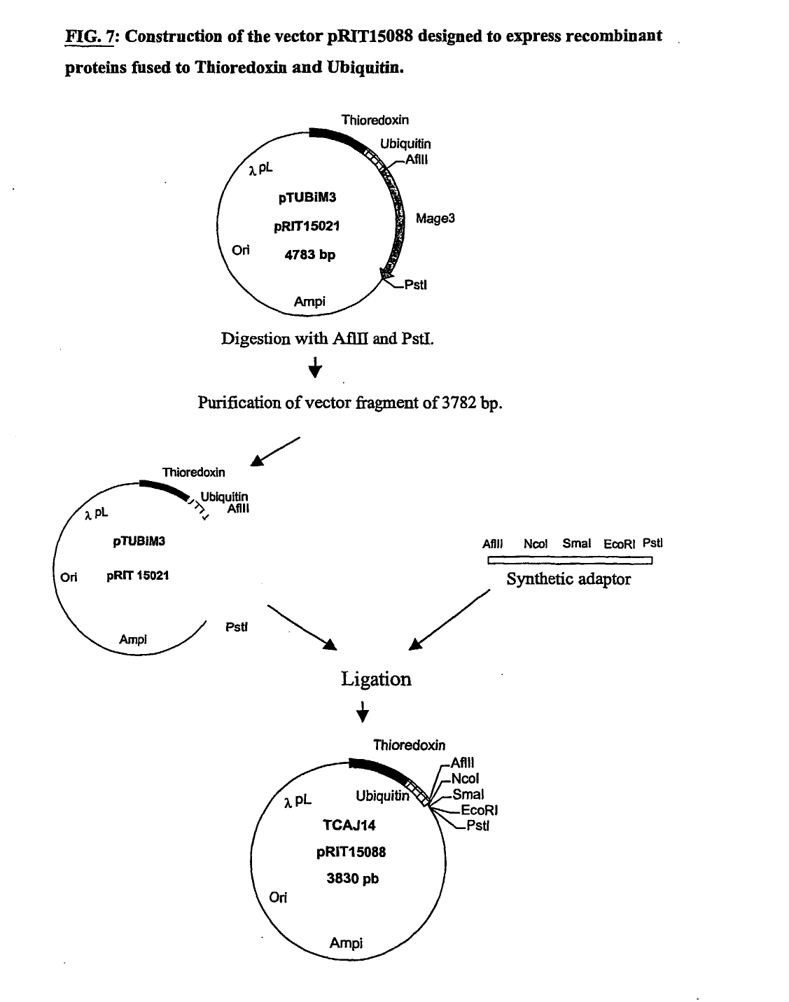

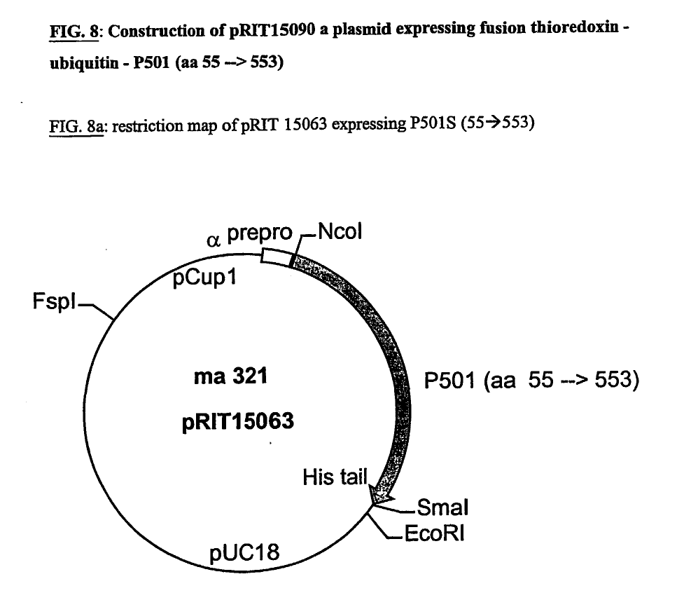

a)—The first step is the construction of the expression vector pRIT 15088 (TCAJ14) (see FIG. 7)

The plasmid pRIT 15021 containing the triple fusion Thioredoxin-Ubiquitin-Mage 3 His under the control of λpL promoter was digested with AflII and PstI restriction enzymes and the restriction fragment AflII-PstI fragment of 3782 bp was purified then ligated with a synthetic adaptor composed of

coding strand: 5′ TTA AGA CTA AGA GGT GGT ATG ACC ATG GTG CCC GGG TGA ATT CCT GCA 3′ and

complementary strand: 5′ GGA ATT CAC CCG GGC ACC ATG GTC ATA CCA CCT CTT AGT C 3′.

This ligation generated the vector pRIT 15088 containing a multiple cloning site useful to fuse heterologous proteins downstream thioredoxin-ubiquitin fusion and realize triple fusions.

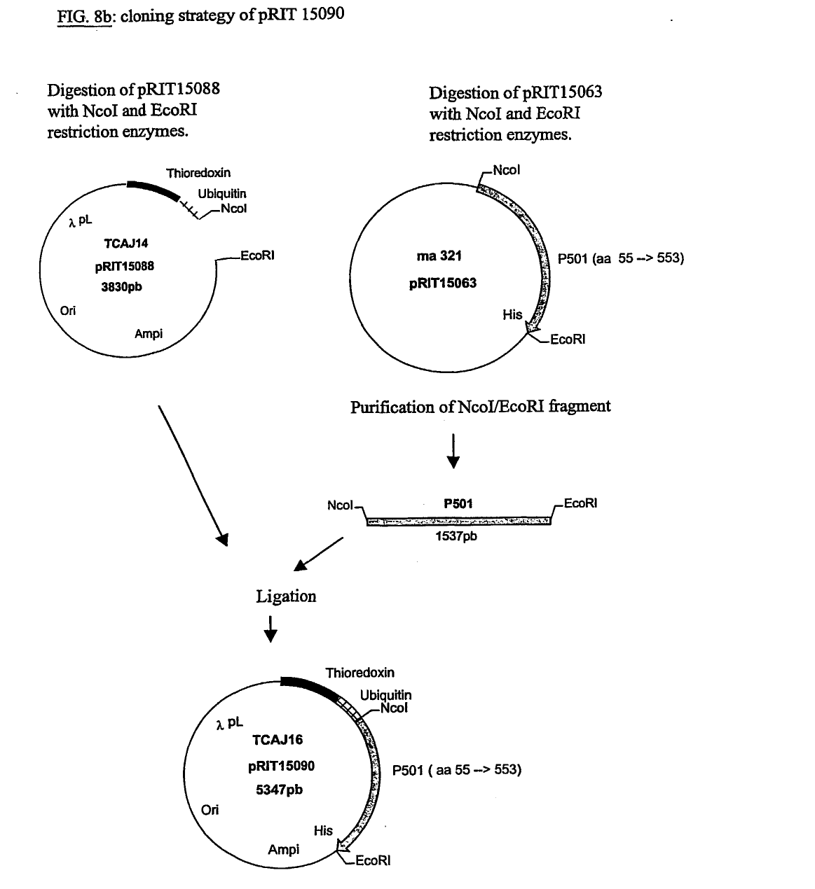

b)—The second step is the construction of the expression vector pRIT 15063 (see restriction map in FIG. 8a). The starting material was the recombinant plasmid p501S, derived from Invitrogen commercial plasmid pcDNA3.1, and containing a 3.4 kb insert between EcoRI and NotI cloning restriction sites. This plasmid contains P501S coding sequence (1662 bp-long) and was obtained from Corixa.

Subcloning of P501S:

A 1569 bp fragment containing nucleotide sequence coding for last 499 aminoacids+68 bp downstream of P501S open reading frame was isolated from p501S plasmid by Nco I digest. After T4 polymerase treatment, the fragment was subcloned in plasmid pUC18 open by PstI and XbaI, T4 polymerase treated, in such a way that NcoI was recovered in N terminal sequence of P501S open reading frame (i.e. amino acid position 55). The plasmid obtained was called pRIT 15061.

Introduction of S. cerevisiae CUP1 promoter and yeast alpha prepro signal sequence:

A PCR fragment containing yeast CUP1 promoter and the yeast alpha prepro signal sequence was obtained by 3 successive PCR steps:

PCR step 1 : amplification of CUP1 promoter with oligonucleotides MDENHE1CUP1 (c 5′ GGA CTA GTC TAG CTA GCT TGC TGT CAG TCA CTG TCA AGA G 3′) and MDECUP1ATG (nc 5′CAT TTT ATG TGA TGA TTG ATT G 3′) on pRIT12471 plasmid as template.

PCR step 2: amplification of alpha preprosignal sequence with oligonucleotides MDEPREPROAT (c 5′CAA TCA ATC AAT CAT CAC ATA AAA TGA GAT TTC CTT CAA TTT TTA CTG CA 3′) and MDESIGNAL2 (nc5′ GCT AGC TCC ATG GCT TCA GCC TCT CTT TTC TCG AG 3′) on pPIC9 plasmid (INVITROGEN) as template.

PCR step 3: association of CUP1 promoter and alpha preprosignal sequence by PCR using fragment obtained by PCR 1 and PCR 2 and oligonucleotides MDENHE1CUP1 and MDESIGNAL2.

After PCR3, amplified fragment was purified, T4 polymerase treated and NcoI digested. Fragment was introduced in plasmid pRIT 15061 between HindIII site, T4 polymerase treated, and NcoI site. This plasmid was called pRIT 15062.

Elongation of the C terminus by HIS tail:

A fragment for HIS tail elongation was obtained by PCR using p501S plasmid as template and oligonucleotides MDE501SAC (c 5′CTG GAG GTG CTA GCA GTG AG 3′) and MDE505HIS (nc 5′CTA GTC TAG AGA ATT CCC CGG GTT AAT GGT GAT GGT GAT GGT GTC CAC CCG CTG AGT ATT TGG CCA AGT CG 3′). The amplified fragment was purified and digested by SacI and EcoRI and introduced between SacI (overlapping aminoacid 439) and EcoRI sites in pRIT 15062 plasmid, restoring correct open reading frame and elongating, in frame, p501S sequence by sequence coding for 2 glycine residues followed by 6 histidine residues followed by a stop codon. Additionally, a SmaI site and EcoRI site have been introduced.

This plasmid was called pRIT 15063 (FIG. 8b).

c)—The third step is the construction of pRIT15090 (TCAJ16), a plasmid expressing the triple fusion thioredoxin-ubiquitin-P501S (aa55→aa553) (see FIG. 8). The plasmid pRIT15088 was digested with NcoI and EcoRI restriction enzymes and ligated with the NcoI-EcoRI fragment of 1537 bp purified from the restriction digestion of plasmid pRIT15063 (ma 321) to give plasmid pRIT15090 (TCAJ16) expressing the triple fusion thioredoxin-ubiquitin-P501S (55→553) His.

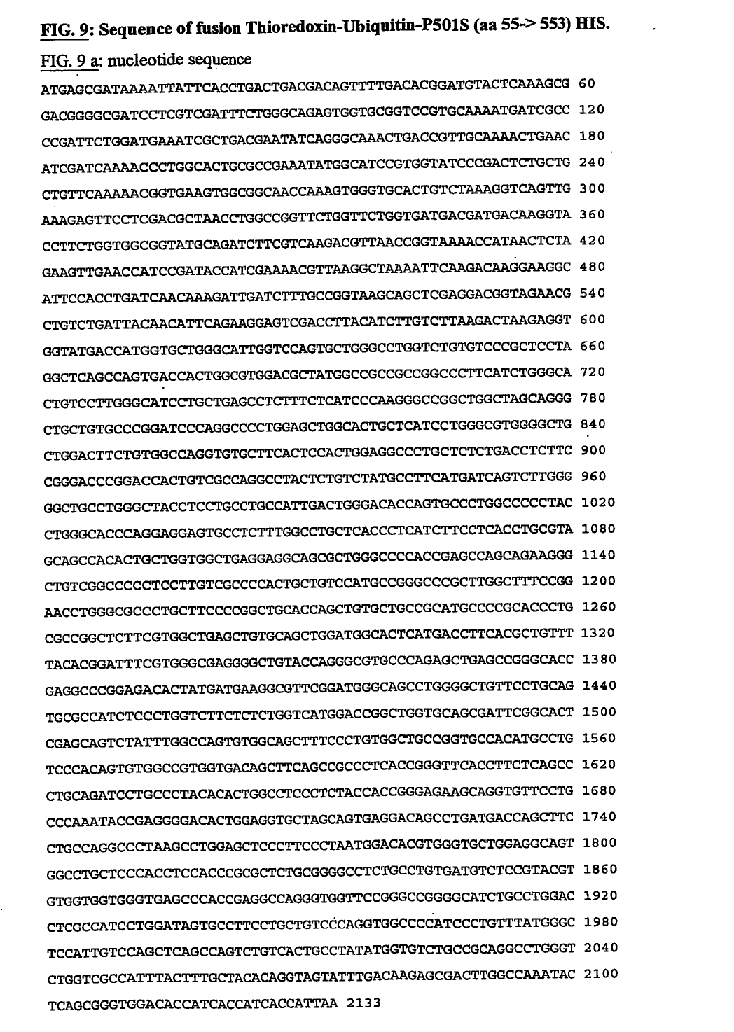

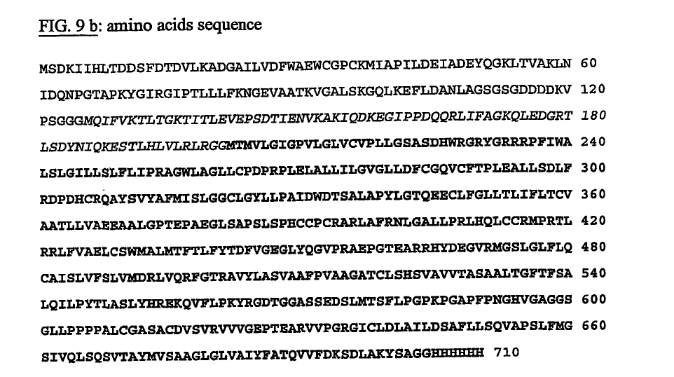

The sequences of the triple fusion thioredoxin-ubiquitin-P501S (55→553) His is given in FIG. 9, with the amino acids sequence described in FIG. 9b (SEQ ID N^{o}4) and the nucleotide coding sequence given in FIG. 9a (SEQ ID N^{o}3). The triple fusion is 710 residues long. After in vivo processing a p501 like protein of 509 amino acids long is generated (in bold in FIG. 9b).

2.—Transformation of E. coli GI724 Strain by pRIT15090 Leading to E. coli B1323 Strain

Plasmid pRIT15090 (ampicillin resistant) was introduced by selection for transformants resistant to ampicillin into E. coli GI724 from Invitrogen, to form E. coli B1323 strain. GI724 contains a engineered cI repressor gene into the bacterial chromosome under control of the tightly-regulated tryptophan promoter, allowing expression of the gene of interest by addition of tryptophan at any temperature. The growth and induction of B1323 strain was carried out as described above.

### EXAMPLE VI

### Construction of an E. coli Strain Expressing the Fusion Protein Thioredoxin-Ubiquitin (55→553) His and Co-expressing the Ubiquitin-specific Endoprotease UBP1

1.—Construction of the Plasmid pRIT15022, a Plasmid Expressing the Ubiquitin-specific Endoprotease UBP1

A plasmid (pRIT15022) derived from pBBR1-2, compatible with pRIT15021 and expressing constitutively the Saccharomyces cerevisiae ubiquitin protease (UBP1) (Kanamycin selection) has been engineered (see FIG. 5).

2.—Transformation of E. coli GI724 Strain to Lead to B1329

The initial E. coli recipient strain GI724 strain was co-transformed by plasmid pRIT15090 (ampicillin resistant) and pRIT15022 (kanamycin resistant) by selection of transformants resistant to both ampicillin and kanamycin.

Plasmid pRIT15022 expresses ubiquitinase UBP1 of Saccharomyces cerevisiae constitutively, allowing cleavage in vivo of the protein of interest from the triple fusion. The growth and induction of B1329 strain was carried out as described above.

### EXAMPLE VII

### Characterisation of the Fusion Protein Thioredoxin-Ubiquitin-P501S (55→553) His and of the in-vivo Processing of the Recombinantly Expressed Fusion Thioredoxin-Ubiquitin-P501S (55→553) His

Frozen cells were thawed and resuspended in PBS buffer. Cells were broken in a cell disrupter One Shot. After centrifugation (20 minutes 16000 g 4° C.) the pellet supernatant and the total cell extract were analysed by SDS-PAGE. Proteins were visualized by Coomassie blue stained gels and identified by Western blot using monoclonal anti P501 PA/G pure 5011 0E3D4G3 3002 (received from Corixa).

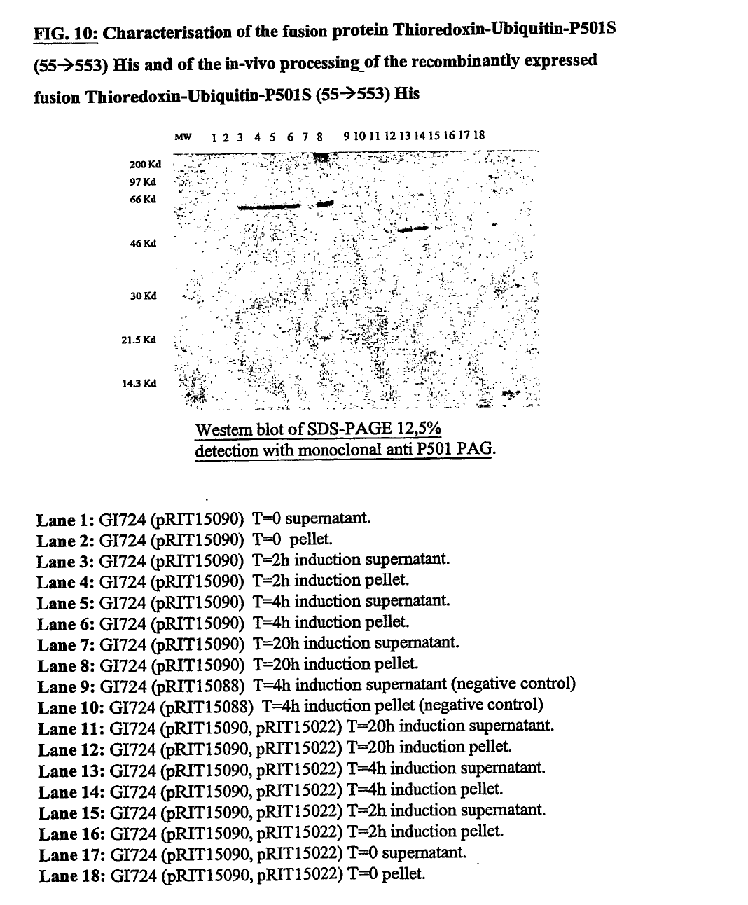

In western blots, a band of ˜50 kd was visualised for the strain B1329, corresponding to the processed P501S (55→553) protein, whereas a band of ˜67 Kd was detected for the strain B1323 (the non processed fusion protein). The results are illustrated in FIG. 10.

### EXAMPLE VIII

### Construction of the E. coli Strain GI724(pRIT15115) Expressing the Fusion Thioredoxin-Ubiquitin-P501S (1-320)-His

1.—Protein Design

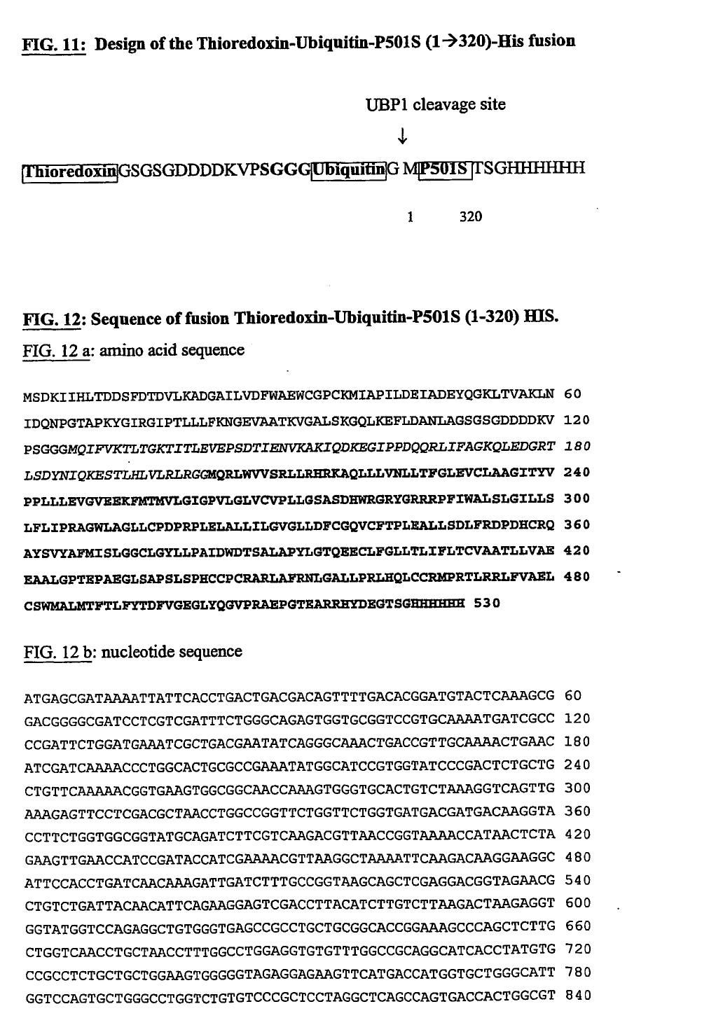

The design of the fusion Thioredoxin-Ubiquitin-P501S (1-320)-His to be expressed in E. coli is described in FIG. 11.

The primary structure of the resulting protein has the sequence described in FIG. 12a (SEQ ID N^{o}5). The coding sequence (see FIG. 12b, SEQ ID N^{o}6) corresponding to the above protein design was placed under the control of λpL promoter from bacteriophage λ in a E. coli expression plasmid in which the P_{L} promoter is tightly regulated by the cI repressor that binds to the operator region in front of the P_{L} promoter.

2.—Cloning Strategy for the Generation of Thioredoxin-Ubiquitin-P501S (1-320) Fusion Protein

Several plasmids were used as starting material for the generation of vector pRIT15115 (TCAJ21) expressing the fusion Thioredoxin-Ubiquitin-P501S (1-320) His:

a plasmid pRIT15021 (pTUbiM3) coding for triple fusion Thioredoxin-Ubiquitin-Mage3 His

a recombinant plasmid expressing P501S, derived from the commercially available Invitrogen plasmid pcDNA3.1, containing a 3.4 Kb insert between EcoRI and NotI cloning restriction sites. This plasmid contains P501S coding sequence (1662 bp long) and was obtained from Corixa.

The cloning strategy included two steps:

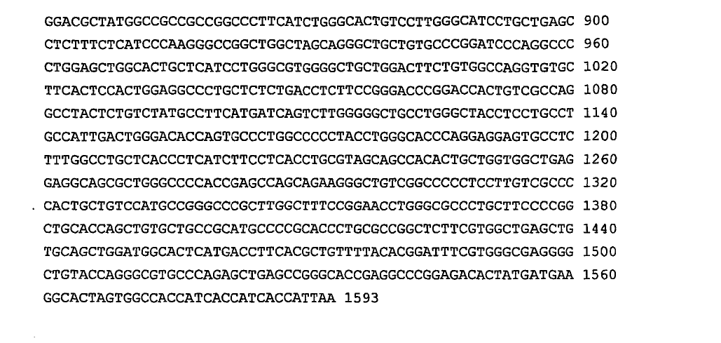

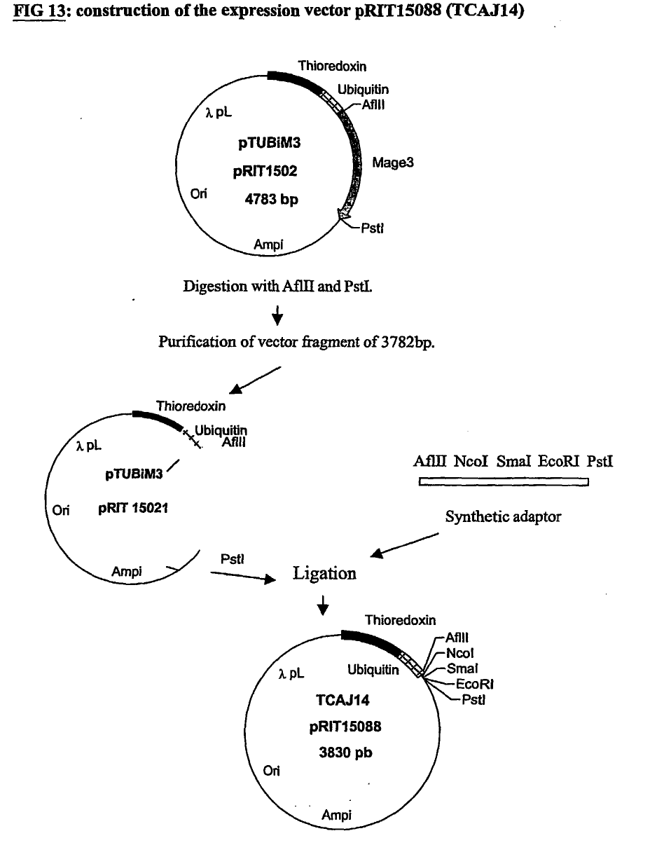

a)—Construction of the expression vector pRIT 15088 (TCAJ14) (see FIG. 13). The plasmid pRIT15021 containing the triple fusion Thioredoxin-Ubiquitine—Mage3 under the control of λpL promoter was digested with AflII and PstI restriction enzymes and the restriction fragment AflII-PstI fragment of 3782 bp was purified, then ligated with a synthetic adaptor composed of coding strand: [5′ TTA AGA CTA AGA GGT GGT ATG ACC ATG GTG CCC GGG TGA ATT CCT GCA 3′] and complementary strand: [5′ GGA ATT CAC CCG GGC ACC ATG GTC ATA CCA CCT CTT AGT C 3′]. This ligation generated the vector pRIT15088 containing a multiple cloning site useful to fuse heterologous proteins downstream Thioredoxin-Ubiquitin fusion and realise triple fusions.

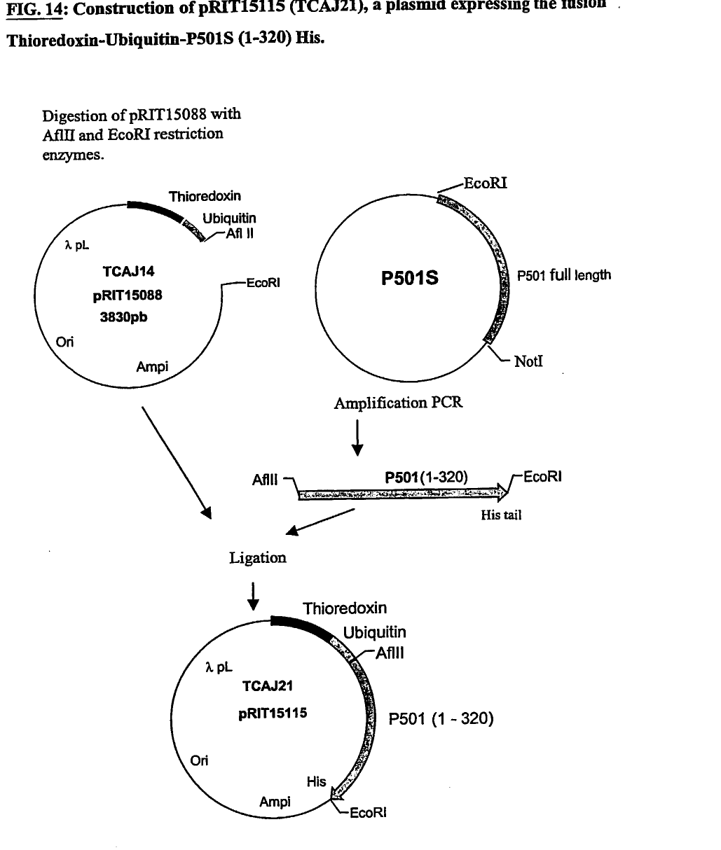

b)—Construction of pRIT5115 (TCAJ21), a plasmid expressing fusion Thioredoxin-Ubiquitin-P501S (1-320) (FIG. 14). The strategy used to generate the plasmid expressing the triple fusion Thioredoxin-ubiquitin-P501S 1→320 consisted in:

Digestion of Plasmid vector pRIT 15088 (TCAJ14) with restriction enzymes AflII and EcoRI.

PCR amplification of coding sequence for Thioredoxin-Ubiquitin-P501S (1-320) in frame with a His tail. The template for the PCR reaction was plasmid P501S from Corixa. The forward primer was the 55-mer oligonucleotide [5′GTC GAC CTT AAG ACT AAG AGG TGG TAT GGT CCA GAG GCT GTG GGT GAG CCG CCT G-3′] and the reverse primer was the 75-mer oligonucleotide [5′ CCG GAA TTC CCC GGG TTA ATG GTG ATG GTG ATG GTG GCC ACT AGT GCC TTC ATC ATA GTG TCT CCG GGC CTC GGT-3′]. The PCR fragment was digested with restriction enzymes AflII and EcoRI to generate the cohesive ends.

Ligation of this PCR fragment with vector pRIT 15088 (TCAJ14) digested with AflII and EcoRI, to generate plasmid pRIT 15115 (TCAJ21).

3.—Obtention of Strain B1356=GI724 (pRIT15115) Expressing Fusion Thioredoxin-ubiquitin-P501S (1-320) His

Plasmid pRIT15115 (ampicillin resistant) was introduced by selection for transformants resistant to ampicillin into E. coli strain GI724 (F-,λ-, lacIq, lacPL8, ampC::Ptrp,cI) from Invitrogen. GI724 contains a engineered cI repressor gene into the bacterial chromosome under control of the tightly-regulated tryptophan promoter, allowing expression of the gene of interest by addition of tryptophan. The growth and induction of B1356 strain was carried out as described previously in Example I, paragraph 4.

### EXAMPLE IX

### Preparation of E. coli Strain Expressing B1395=GI724 (pRIT15139) Expressing P501S (1-320) Without Fusions Partners

1.—Construction of Plasmid pRIT15139 (TCAJ23) a Control Counterpart Expressing P501S (1-320) Without Fusions Partners Thioredoxin and Ubiquitin

The starting materials were:

Plasmid vector pAL781 (Invitrogen) used as a control vector for expression of protein downstream λpL promoter. It does not contain thioredoxin.

Recombinant plasmid P501S, derived from commercial plasmid pcDNA3.1 (Invitrogen) containing a 3,4 Kb insert between EcoRI and NotI cloning restriction sites. This plasmid contains the P501S full length coding sequence (1662 bp long) and was obtained from Corixa.

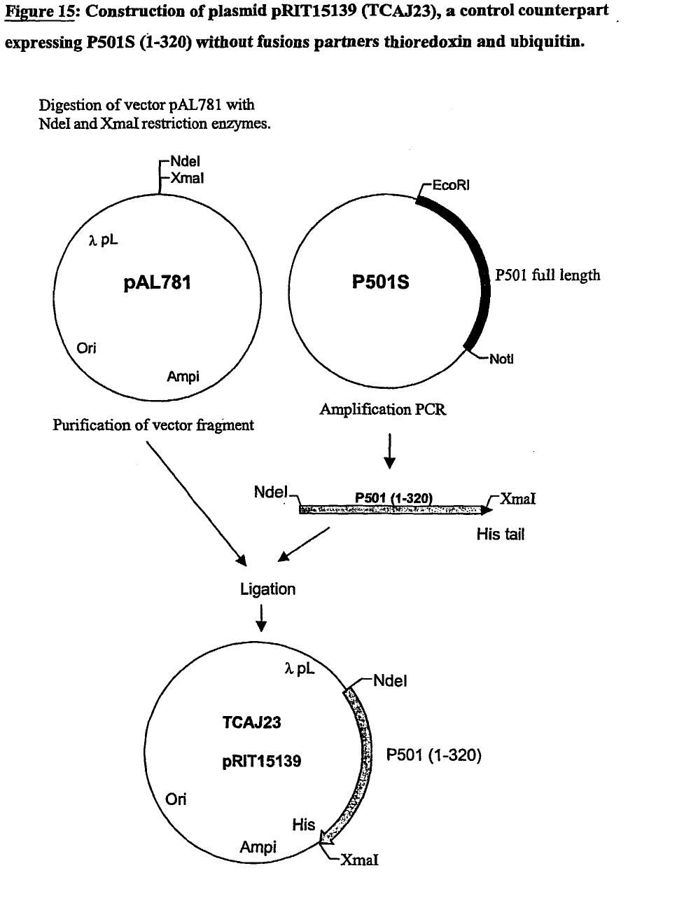

The cloning strategy is outlined in FIG. 15.

a)—Digestion of vector pAL781 by restriction enzymes NdeI and XmaI.

b)—PCR amplification of P501S (1-320) coding sequence in frame with a His tail. The template for the PCR reaction was plasmid P501S from Corixa. The forward primer was the 40-mer oligonucleotide [5′GGA ATT CCA TAT GGT CCA GCG TCT GTG GGT GAG CCG CCT G 3′] and the reverse primer was the 75-mer oligonucleotide [5′ CCG GAA TTC CCC GGG TTA ATG GTG ATG GTG ATG GTG GCC ACT AGT GCC TTC ATC ATA GTG TCT CCG GGC CTC GGT-3′]. The PCR fragment was digested with NdeI and XmaI restricion enzymes to generate the cohesive ends.

c)—Ligation of the PCR fragment with vector pAL781 digested with NdeI and XmaI, to generate plamid pRIT15139 (TCAJ23).

2.—Generation of a Control Counterpart Strain B1395=GI724 (pRIT15139) Expressing P501S 1->320 Without Fusions Partners.

Plasmid pRIT15139 (ampicillin resistant) was introduced into E. coli GI724 (F-,λ, lacIq,lacPL8,ampC::Ptrp, cI) by selection for transformants resistant to ampicillin. The growth and induction of B1395 strain was carried out as described previously in Example I, paragraph 4.

### EXAMPLE X

### Preparation of E. coli Strain Expressing B1372=GI724 (pRIT15115, pRIT15022) Expressing Fusion Thioredoxin-ubiquitin-P501S 1->320 and Ubiquitinase UBP1

1.—Construction of the Plasmid pRIT15022 Expressing Constituvely Ubiquitinase UBP1 from Saccharomyces cerevisiae.

see FIG. 5 and Example III, §1.

2.—Generation of Strain B1372=GI724 (pRIT15115, pRIT15022) Expressing Fusion Thioredoxin-ubiquitin-P501S 1->320 and Ubiquitinase UBP1.

E. coli strain G1724 (F-,λ-, lacIq, lacPL8, ampC::Ptrp,cI) was co-transformed with plasmid pRIT15115 (ampicillin resistant) and pRIT15022 (kanamycin resistant) by selection of transformants resistant to ampicillin and kanamycin. Plasmid pRIT15022 expresses ubiquitinase UBP1 of Saccharomyces cerevisiae constitutively, allowing cleavage in vivo of the protein of interest from the triple fusion. The growth and induction of B1372 strain was carried out as described previously in Example I, paragraph 4.

### EXAMPLE XI

### Characterisation of the Fusion Protein Thioredoxin-Ubiquitin-P501S (1→320) His and of the in-vivo Processing of the Recombinantly Expressed Fusion Thioredoxin-Ubiquitin-P501S (1→320) His

1.—Preparation of Extracts/Analysis on Coomassie Stained SDS-polyacrylamide 12,5% Gels and Western Blots

Frozen cells were thawed and resuspended in PBS buffer. Cells were broken in a cell disrupter One Shot. After centrifugation (20 minutes 16000 g 4° C.) pellet, supernatant and total extract were analysed by SDS-PAGE. Proteins were visualized on Coomassie blue stained gels and identified by western blot using anti his tail monoclonal antibodies (Pentahis from Qiagen).

The experimental approach described above has allowed a comparison of the expression pattern of P501S 1→320 alone, P501S 1→320 tri-fusion form and P501S 1→320 processed in vivo using the same genetic system (pL promoter, tryptophan induction, 30° C.).

2.—Results and Conclusions

For the strain B1356=GI724 (pRIT15115) a band of approximately 50 KD corresponding to the triple fusion Thioredoxin-Ubiquitin-P501S 1->320 was slightly visible in Coomassie stained gels, distributed in supernatant and pellet, recognized in western blot by anti Pentahis monoclonal (Qiagen).

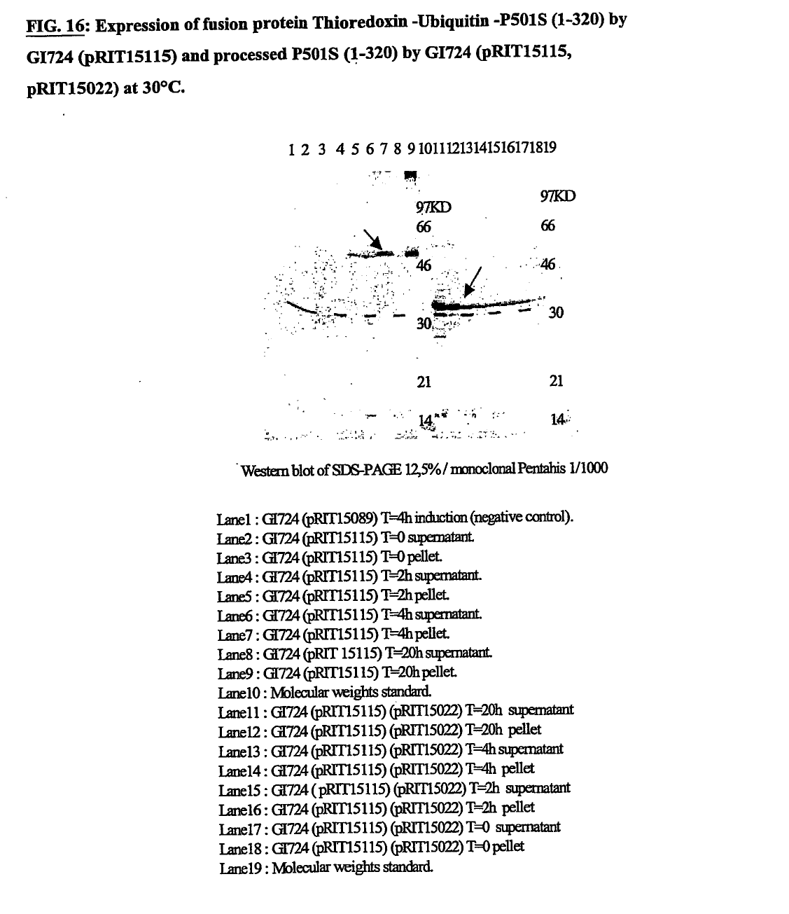

In order to assess the in vivo cleavage of the triple fusion, strain B1372=GI724 (pRIT15115, pRIT15022) simultaneously expressing the triple fusion and Ubiquitinase UBP1, was expressed. Two resulting fragments were obtained, visible in Coomassie gel and in western blot (FIG. 16), found at approximately 32 KD and 25 KD. The 32 KD band, highly present in supernatant, represents the processed P501S (1->320) protein as demonstrated by western blot and the band of 25 KD corresponds with the cleaved fusion partners thioredoxin and ubiquitin.

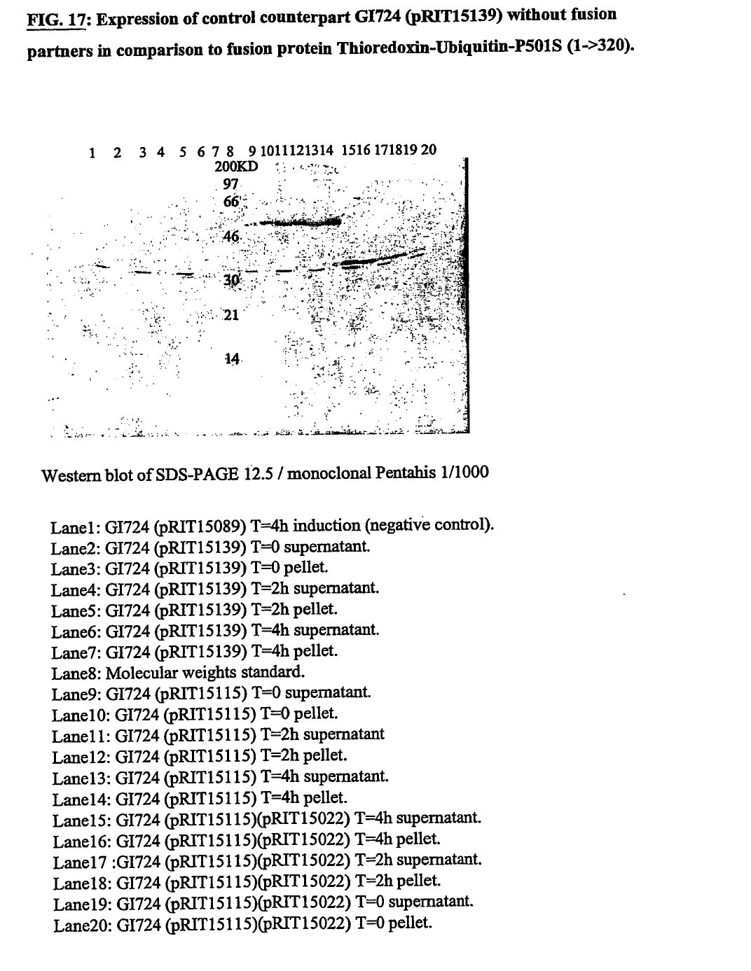

As to the control counterpart strain B1395=GI724 (pRIT15139) expressing the P501S (1→320) without fusion partners, no recombinant protein could be identified neither in stained gels, nor in western blot by Pentahis monoclonal (FIG. 17). In this case, the efficient in vivo processing of the triple fusion protein went along with an important increase in yield of protein P501S (1→320), in comparison to the counterpart expressed as a single protein which was not expressed at all, and led to a highly soluble product.

### EXAMPLE XII

### Construction of the E. coli Strain Strain B1357=GI724 (pRIT15106) Expressing Fusion Thioredoxin-ubiquitin-ProtD1/3-E7

1.—Protein Design

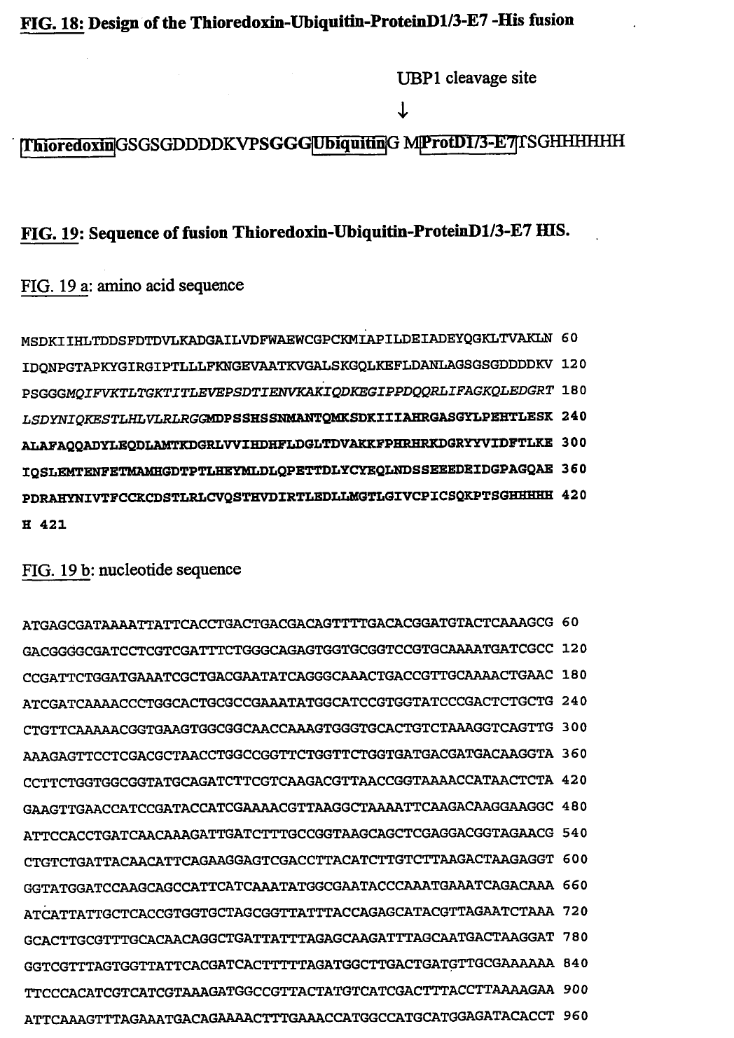

The design of the fusion Thioredoxin-Ubiquitin-ProtD1/3-E7-His to be expressed in E. coli is described in FIG. 18.

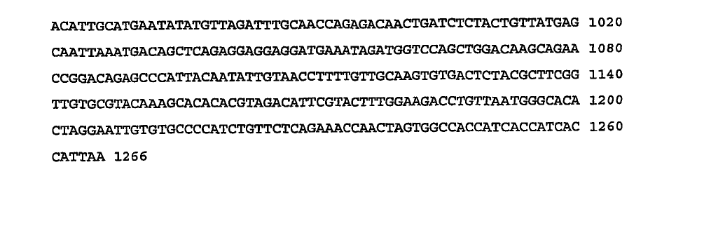

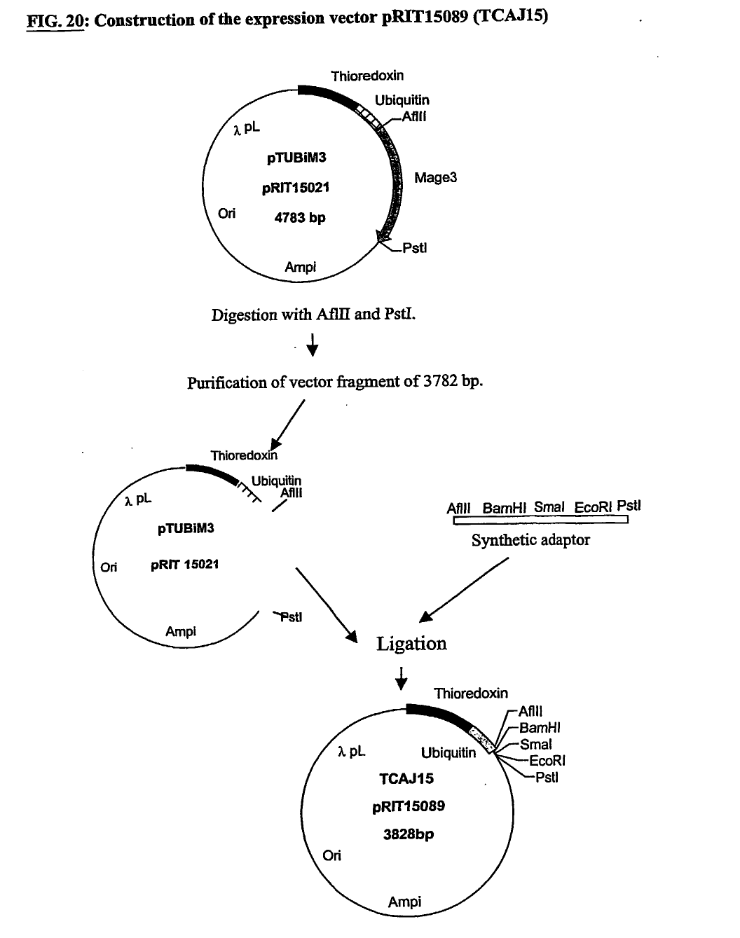

The primary structure of the resulting protein has the sequence described in FIG. 19a (SEQ ID N^{o}7). The coding sequence (see FIG. 19b, SEQ ID N^{o}8) corresponding to the above protein design was placed under the control of λpL promoter from bacteriophage λ in a E. coli expression plasmid in which the P_{L} promoter is tightly regulated by the cI repressor that binds to the operator region in front of the P_{L} promoter.

2.—Cloning Strategy for the Generation of Thioredoxin-Ubiquitin-ProteinD1/3-E7 Fusion Protein

The starting materials are:

plasmid pRIT15021 (pTUbiM3) coding for triple fusion Thioredoxin-Ubiquitin-Mage3

plasmid pRIT14501 (TCA308) bearing the coding sequence for fusion protein ProtD1/3-E7, elongated with a polyhistidine tail at the C-terminal (WO 99/10375).

The cloning strategy included two steps:

a)—Construction of the expression vector pRIT15089 (TCAJ15) (FIG. 20). The plasmid pRIT15021 containing the triple fusion Thioredoxin-Ubiquitine-Mage3 under the control of λpL promoter was digested with AflII and PstI restriction enzymes and the restriction fragment AflII-PstI fragment of 3782 bp was purified then ligated with a synthetic adaptor composed of coding strand: [5′ TTA AGA CTA AGA GGT GGT ATG GAT CCT GCC CGG GTG AAT TCC TGC A 3′] and complementary strand: [5′GGA ATT CAC CCG GGC AGG ATC CAT ACC ACC TCT TAG TC 3′]. This ligation generated the vector pRIT15089 containing a multiple cloning site useful to fuse heterologous proteins downstream Thioredoxin-Ubiquitin fusion and generate triple fusions.

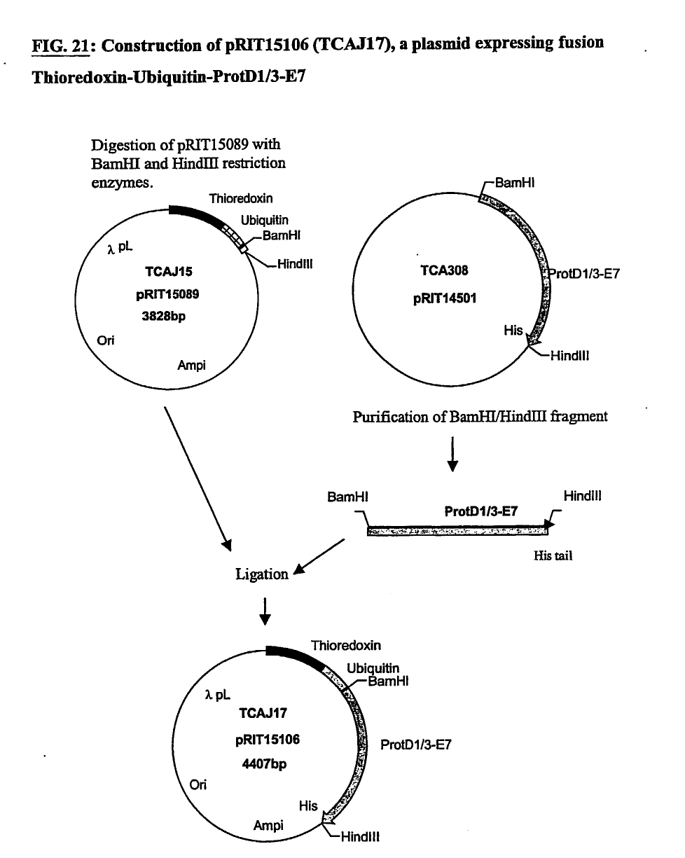

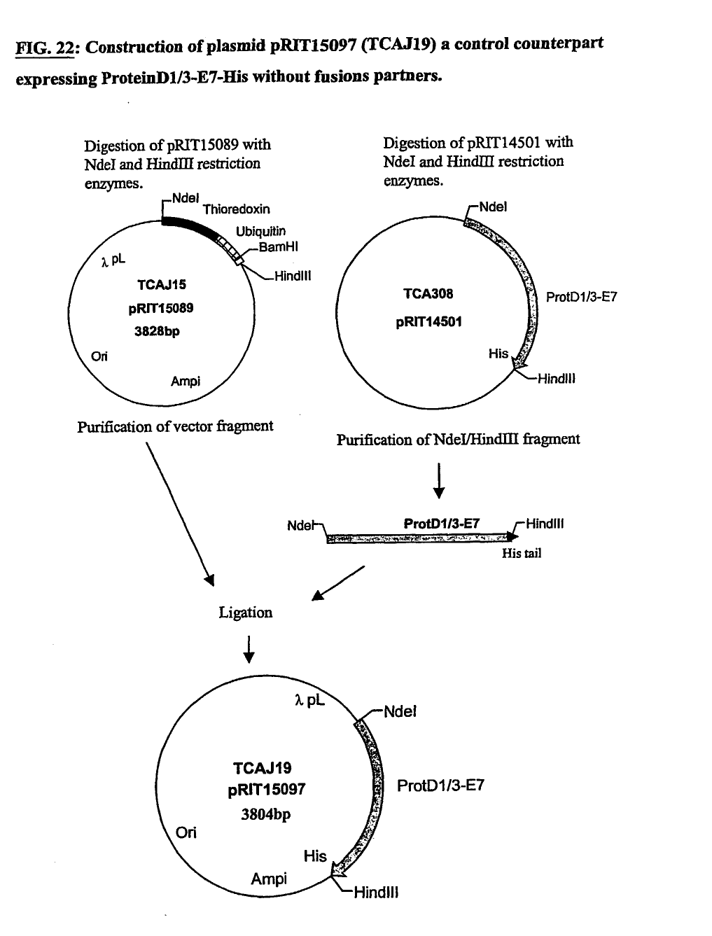

b)—Construction of pRIT15106 (TCAJ17), a plasmid expressing fusion thioredoxin-ubiquitin-ProtD1/3-E7 (see FIG. 21).

Plasmid vector pRIT15089 was digested with BamHI and HindIII restriction enzymes and ligated with the BamHI-HindIII fragment of 674 bp, purified from the restriction digestion of plasmid pRIT14501 (TCA308) to give plasmid pRIT15106 (TCAJ17) expressing triple fusion thioredoxin-ubiquitin-ProtD1/3-E7.

3.—Obtention of Strain B1357=GI724 (pRIT15106) Expressing Fusion Thioredoxin-Ubiquitin-ProtD1/3-E7 His

Plasmid pRIT15106 (ampicillin resistant) was introduced by selection for transformants resistant to ampicillin into E. coli train GI724 (F-,λ-,lacIq,lacPL8, ampC::Ptrp,cI ) from Invitrogen. GI724 contains an engineered cI repressor gene into the bacterial chromosome under the control of the tightly-regulated tryptophan promoter, allowing expression of the gene of interest by addition of tryptophan. The growth and induction of B1357 strain was carried out as described previously in Example I, paragraph 4.

### EXAMPLE XIII

### Preparation of E. coli Strain Expressing B1344=GI724 (pRIT15097) Expressing ProteinD1/3-E7 His Without Fusions Partners

1.—Construction of Plasmid pRIT15097 (TCAJ19) a Control Counterpart Expressing ProteinD1/3-E7-His Without Fusions Partners

The starting materials are:

Plasmid vector pRIT15089 (TCAJ15)

Plasmid pRIT 14501 (TCA308) bearing the coding sequence for ProtD1/3-E7-His

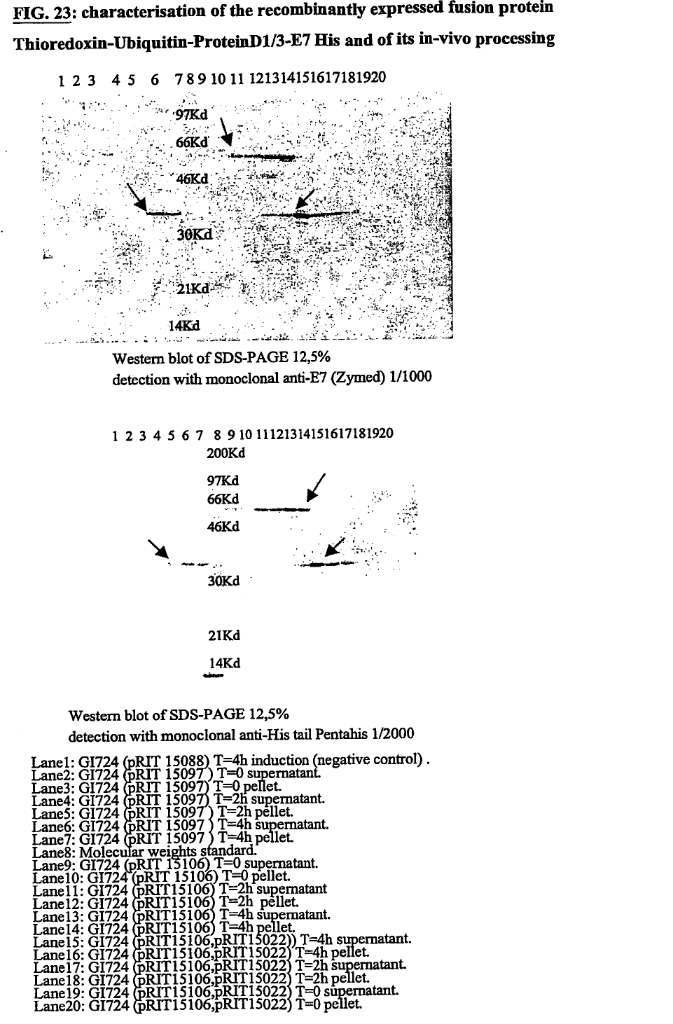

The cloning strategy is outlined in FIG. 22. Plasmid vector pRIT15089 was digested with NdeI and HindIII restriction enzymes and ligated with the NdeI-HindIII fragment of 677 bp purified from the restriction digestion of plasmid pRIT 14501. This gives rise to plasmid pRIT15097 (TCAJ19), expressing ProtD1/3-E7-His.

2.—Generation of a Control Counterpart Strain B1344=GI724 (pRIT15097) Expressing ProteinD1/3-E7 Without Fusions Partners.

Plasmid pRIT15097 (ampicillin resistant) was introduced into E. coli GI724 (F,λ-,lacIq,lacPL8,ampC::Ptrp,cI) by selection for transformants resistant to ampicillin (100 μgr/ml). The growth and induction of B1344 strain was carried out as described previously, in Example I, paragraph 4.

### EXAMPLE XIV

### Preparation of E. coli Strain Expressing B1347=GI724 (pRIT15106, pRIT15022) Expressing Fusion Thioredoxin-ubiquitin-ProteinD1/3-E7 His and UBP1

1.—Construction of the Plasmid pRIT15022 Expressing Constituvely Ubiquitinase UBP1 from Saccharomyces cerevisiae.

see FIG. 5 and Example III, §1.

2.—Generation of Strain B1347=GI724 (pRIT15106, pRIT15022) Expressing Fusion Thioredoxin-ubiquitin-ProteinD1/3-E7 His and Ubiquitinase UBP1.

E. coli strain G1724 (F-,λ-, lacIq, lacPL8, ampC::Ptrp,cI) was co-transformed with plasmid pRIT15106 (ampicillin resistant) and pRIT15022 (kanamycin resistant) by selection of transformants resistant to ampicillin and kanamycin. Plasmid pRIT15022 expresses ubiquitinase UBP1 of Saccharomyces cerevisiae constitutively, allowing cleavage in vivo of the protein of interest from the triple fusion. The growth and induction of B1347 strain was carried out as described previously in Example I, paragraph 4.

### EXAMPLE XV

### Characterisation of the Fusion Protein Thioredoxin-Ubiquitin-ProteinD1/3-E7 His and of the in-vivo Processing of the Recombinantly Expressed Fusion Thioredoxin-Ubiquitin-ProteinD1/3-E7 His

1.—Preparation of Extracts/Analysis on Coomassie Stained SDS-polyacrylamide 12,5% Gels and Western Blots

Frozen cells were thawed and resuspended in PBS buffer. Cells were broken in a cell disrupter One Shot. After centrifugation (20 minutes 16000 g 4° C.) pellet supernatant and total extract were analysed by SDS-PAGE. Proteins were visualized on Coomassie blue stained gels and identified by western blot using anti HPV16 E7 (Zymed) and anti his tail monoclonal antibodies (Pentahis de Qiagen). This is shown on FIG. 23.

The experimental approach described above has allowed a comparison of the expression pattern of ProtD1/3-E7 alone, ProtD1/3-E7 tri-fusion form and PrrotD1/3-E7 processed in vivo using the same genetic system (pL promoter, tryptophan induction, 30° C.).

2.—Results and Conclusions

For the strain control counterpart B1344=GI724 (pRIT 15097) expressing the ProtD1/3-E7 without fusion partners, a major band of approximately 33KD was detected sligthly visible in Coomassie gels and distributed half in the supernatant and half in the pellet.

For the strain B1357=GI724 (pRIT 15106) a band of approximately 56 KD corresponding to the triple fusion Thioredoxin-Ubiquitin-ProtD1/3-E7 His was visible in Coomassie gels and identified in western blots using anti E7 (Zymed) and anti pentahis (Qiagen) monoclonal antibodies.

To test for in vivo cleavage of the triple fusion, strain B1347=GI724 (pRIT15106, pRIT15022) simultaneously expressing the triple fusion and UBP1, was expressed. Two resulting fragments were obtained, highly visible in Coomassie gels found at approximately 33 KD and 25 KD. The 33 KD band, mainly present in supernatant represents the processed ProtD1/3-E7 protein as demonstrated by western blot (FIG. 23) and the band of 25 KD corresponds with the cleaved fusion partners Thioredoxin and Ubiquitin.

### EXAMPLE XVI

### Construction of the Compatible Plasmid Expressing Ubiquitinase (UBP1) from Saccharomyces cerevisiae Under the Controle of pTRC Promoter

1.—Construction of the Compatible Ubiquitinase Expression Plasmid pRIT15113 (TCAJ20) Inducible with IPTG.

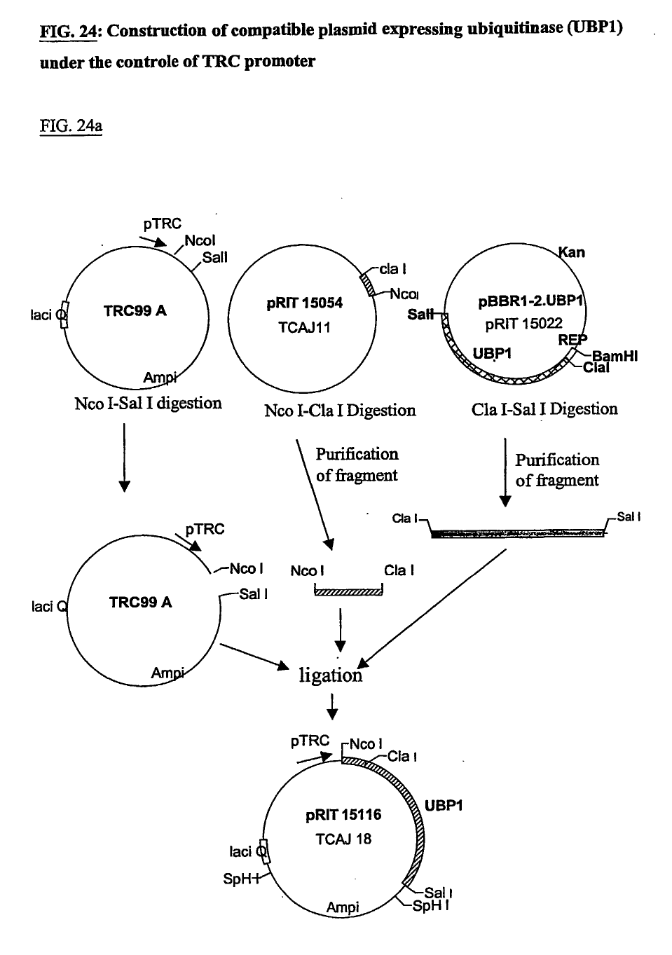

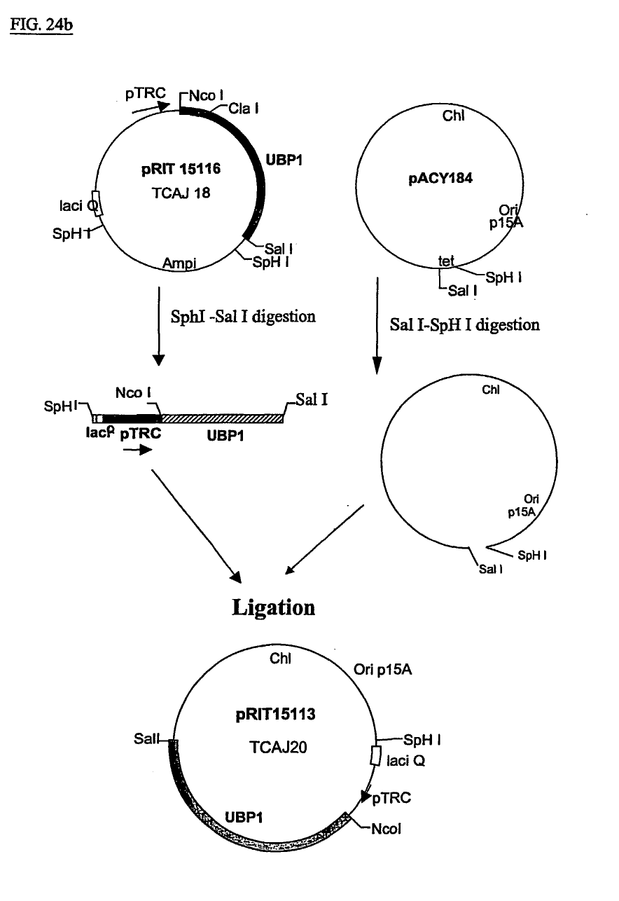

The outline of the different steps required for the obtention of the above plasmid is presented in FIG. 24. The first step consists in the introduction of ubiquitinase coding sequence in vector TRC99A under the controle of TRC promoter (purchased from Pharmacia, cat n^{o} 27-5007-01) (FIG. 24a). pRIT15054 (TCAJ11) is an intermediate construct where a 113 bp NcoI-ClaI fragment of UBP1 (generated by PCR) has been cloned in pCRII-Topo (Invitrogen cat n^{o} K4650-01). As a result of this manipulation, a NcoI cloning site has been created which overlaps the UBP1 ATG codonn. As a second step, the fragment SpH I-Sal I from TCAJ18 (pRIT1516) has been transferred into the compatible vector pACY184 (purchased from New Egland BioLabs catalog n^{o}. E4152S) (FIG. 24b).

2.—Preparation of E. coli Strain B1500=G1724 (pRIT15106,pRIT15113) Expressing Fusion Thioredoxin-ubiquitin-ProtD1/3-E7-his and Inducible UBP1.

The plasmid pRIT15113 was transformed into the recipient strain expressing the triple fusion thioredoxin-ubiquitin-ProtD1/3 (see example XII) to give E. coli strain B1500=GI724 (pRIT15106, pRIT15113).

This strain was characterised for the expression of the trimera fusion and for the in vivo processing of the trimera at two different times after induction. The efficient cleavage of the trimera ProtD1/3-E7 occured at the two examined conditions. Additionally, higher level of the cleaved protein can be observed when the processing cleavage does not occur cotranslationally that is to say, when the induction of the UBP1 enzyme is done later during fermentation, after the induction of ProtD1/3-E7 triple fusion, once the whole trimera has been expressed.

Conclusions

We have demonstrated that the system developed according to the invention is leading to the production of a recombinant polypeptide of interest, with improved solubility and better folding. Better solubility likely to be associated with a better folding is defined experimentally by the lower proportion of the recombinant product found associated to the inclusion bodies, as assessed either by SDS-PAGE analysis of the protein supernatant and pellet, followed by Coomassie blue staining, scanning and analysis of the scans by imaging densitometry; or by the increased possibility to solubilise the recombinant product from the inclusion bodies using denaturing agents. In-vivo cleavage of the polypeptide of interest by ubiquitinase has been achieved both in conditions where ubiquitinase is under the control of a constitutive and inducible promoter.

We have demonstrated that the method as described is capable of generating in flask culture expression yields that are compatible with industrial upscaling of the system, allowing for the production of protein of industrial importance. Indeed scaled-up, parameterized cultures may generate even better expression results.

Lastly, the expression vector could be engineered to allow versatile cloning downstream from the fusion partners and be tested for various protein expression.

1. A DNA sequence encoding a triple fusion protein comprising ubiquitin fused between thioredoxin and a polypeptide of interest; and optionally comprising an affinity tag at its carboxy-terminus.

2. A DNA sequence encoding a triple fusion protein according to claim 1, wherein the polypeptide of interest is a tumor associated antigen or a derivative thereof.

3. A DNA sequence encoding a fusion protein as claimed in claims 1 or 2, wherein the tumor associated antigen is selected from the group comprising Mage, PS108, P501S, Cripto, Prame, C74_{—}39, C76_{—}1 and protase.

4. A DNA sequence encoding a triple fusion protein as claimed in any of claims 1 to 3, wherein the affinity tag is selected from the group comprising Histidine tag of at least four histidine residues, or C-Lyta tag.

5. An expression vector containing a DNA sequence as claimed in claims 1 to 4.

6. A bacterial host cell transformed with a DNA sequence of any of claims 1 to 4.

7. A bacterial host cell according to claim 6 additionally co-transformed with a DNA sequence encoding a ubiquitin-specific endoprotease.

8. A bacterial cell host according to claim 7 wherein the ubiquitin-specific protease is under the control of a constitutive promotor.

9. A bacterial host cell according to claims 7 to 8 wherein the ubiquitin-specific endoprotease is UBP1 from Saccharomyces cerevisae.

10. A bacterial host cell of any of claims 6 to 9 which is E. coli.

11. A method of producing a recombinate polypeptide of interest with an authentic amino-terminus, comprising:
(a) culturing a bacterial host cell of any the claims 7 to 9 under conditions which allow for the co-expression of the triple fusion encoded by the DNA of any of claims 1 to 4 and of the ubiquitin-specific endoprotease and
(b) recovering the recombinant polypeptide of interest directly from the bacterial host cells after it has been subjected to the action of the ubiquitin-specific endoprotease in vivo.

12. The method of claim 11 wherein the ubiquitin-specific endoprotease is UBP1 from Saccharomyces cerevisae.

13. The method of claim 11 or 12 wherein the bacterial host cell is E. coli.
